(19) United States
(12) Patent Application Publication
(10) Pub. No.: US 2025/0346941 A1
(43) Pub. Date: Nov. 13, 2025

(54) SENSOR FOR ANALYTE DETECTION, METHODS OF CONSTRUCTION, AND METHODS OF USE THEREOF

(71) Applicant: VIRGINIA TECH INTELLECTUAL PROPERTIES, INC., Blacksburg, VA (US)

(72) Inventors: Azahar Ali, Christiansburg, VA (US); Katharine Knowlton, Blacksburg, VA (US); Shannon Chick, Blacksburg, VA (US)

(21) Appl. No.: 19/202,105

(22) Filed: May 8, 2025

Related U.S. Application Data
(60) Provisional application No. 63/644,551, filed on May 9, 2024.

(51) Int. Cl. C12Q 1/32; C12Q 1/00; G01N 27/327

(52) U.S. Cl. CPC C12Q 1/32; C12Q 1/005; G01N 27/3271

(57) ABSTRACT

In one aspect, the disclosure relates to a system and an apparatus comprising electrodes and a support. The disclosure also relates to methods for measuring an analyte in a biological sample using any one of the systems disclosed herein. Also disclosed herein are methods for fabricating a sensor. This abstract is intended as a scanning tool for purposes of searching in the particular art and is not intended to be limiting of the present disclosure.

### CROSS-REFERENCE TO RELATED APPLICATIONS

This Application claims the benefit of and priority to U.S. Provisional Application No. 63/644,551, filed on May 9, 2024 which is incorporated herein by reference in its entirety.

### BACKGROUND

Precision livestock farming is one method of improving the productivity, health, and well-being of animals. The use of advanced diagnostic tools can be a good strategy for the management of livestock operations. Early detection of diseases in livestock can reduce the overall cost and time of treatment and managing the effects of the disease. With many diseases prevalent in livestock providing little to no visual indication, alternate methods of detection that are quick, convenient, and low-cost are needed.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A is a schematic diagram illustrating an example of a Keto-sensor and showing the connectors, working electrode (WE), counter electrode (CE), and reference electrode (RE), in accordance with various embodiments of the present disclosure.

FIG. 1B shows a scanning electron microscopy (SEM) image of an enzyme that was immobilized onto the functionalized graphene/WE, in accordance with various embodiments of the present disclosure.

FIG. 1C shows a visual representation of how decreased glucose levels lead to fat metabolism and increased βHB levels in dairy cattle, in accordance with various embodiments of the present disclosure.

FIG. 1D shows a schematic diagram illustrating the creation of the WE for the ketosis sensor (Keto-sensor), in accordance with various embodiments of the present disclosure. Functionalized graphene coats the carbon substrate of the screen-printed electrode. Once the graphene was dried onto the surface of the substrate, then an N-Ethyl-N′-(3-dimethylaminopropyl) carbodiimide (EDC)-N-hydroxysuccinimide (NHS) (EDC-NHS) solution was added and allowed to sit in a humid chamber. After 4 hours, the excess EDC-NHS was washed from the surface and the beta-hydroxybutyrate dehydrogenase (βHBD) enzyme solution was added and allowed to sit in the humid chamber for up to 12 hours. The stability of the enzyme was increased by treating the sensor surface with a stabilizer, i.e., glycerol. When testing the sensor, the beta-hydroxybutyrate in the sample reacted with the βHBD.

FIG. 1E shows a visual representation of a dairy cow that can produce gallons of milk that can be tested for beta-hydroxybutyrate as a ketosis biomarker, in accordance with various embodiments of the present disclosure.

FIGS. 2A-2C show examples of SEM images for a screen-printed sensor without modification (FIG. 2A), a graphene sensor without enzyme (FIG. 2B), and a sensor with graphene nanosheets along with enzyme or Keto-sensor (FIG. 2C), in accordance with various embodiments of the present disclosure.

FIGS. 2D-2F show examples of energy-dispersive X-ray spectroscopy (EDS) mapping of the WE of the Keto-sensor showing nitrogen distribution, in accordance with various embodiments of the present disclosure.

FIG. 2G shows a graph of weight distribution of different elements found on the WE of the Keto-sensor, in accordance with various embodiments of the present disclosure.

FIG. 2H shows a table with the weight distribution of the different elements found on the WE of the Keto-sensor, in accordance with various embodiments of the present disclosure.

FIG. 3A shows an example of cyclic voltammetry (CV) of the Keto-sensor with and without γHB (1 mM) concentration, in accordance with various embodiments of the present disclosure. For the CV test, the PBS solution was mixed into a 5 mM concentration of a [Fe(CN)₆]^{3-/4-} redox mediator.

FIG. 3B shows an example of a CV comparison for the S-sensor, G-sensor, and Keto-sensor in the presence of βHB (1 mM) concentration, in accordance with various embodiments of the present disclosure.

FIG. 3C shows an example of a scan rate study for the Keto-sensor wherein the scan rate was varied from 0.5 V/s to 1.0 V/s, in accordance with various embodiments of the present disclosure. As the scan rate increases, the current peak increases.

FIG. 3D shows an example of a calibration plot of scan rate studies comparing the peak current during different scan rates, in accordance with various embodiments of the present disclosure.

FIGS. 4A-4F illustrate examples of the sensing performance of a screen-printed sensor (S-sensor, FIG. 4A-4B) and a screen-printed sensor with graphene nanosheets (G-sensor, FIG. 4C-4D), and Keto-sensor (FIG. 4E-4F) at different concentrations of βHB: 0.01 μm, 1.00 μm, 0.10 mM, 0.25 mm, 0.50 mm, 1.00 mm, and 3.00 mm, in accordance with various embodiments of the present disclosure. 4A, 4C, and 4E show the CV responses for the S-sensor, G-sensor and Keto-sensor, respectively, with increasing concentration of βHB in buffer solution. FIGS. 4B, 4D, and 4F are the sensor calibration plots for S-sensor, G-sensor, and Keto-sensor, respectively. For all three sensors, the increase in βHB concentration shows an increase in peak current values. Data shown in FIGS. 4B, 4D, and 4F was produced by three repeated measurements of biologically independent concentration of targets. Error bars are calculated by taking the standard deviations of the three repeated measurements. All dilution studies were conducted at room temperature and used the same βHB serial dilutions and buffer solution.

FIG. 5A shows an example of a comparison plot of the calibration plots for the S-sensor, G-sensor, and Keto-sensor, in accordance with various embodiments of the present disclosure.

FIG. 5B shows an example of a CV graph of a Keto-sensor testing deionized water, fetal bovine serum, and fetal bovine serum spiked with 3 mM of βHB, in accordance with various embodiments of the present disclosure. The addition of βHB to the serum increases the peak values recorded during CV.

FIG. 5C shows an example of a chronoamperometry graph showing long-term and continuous sensing of 3 mM βHB, 1 mM βHB, and buffer solutions, in accordance with various embodiments of the present disclosure. Continuous measurements were run for a duration of 10 min.

FIG. 5D shows an example of a chronoamperometry graph for a selectivity study on the Keto-sensor, in accordance with various embodiments of the present disclosure. Different solutions of urea and glucose, with and without βHB, were added to the sensor and chronoamperometry was performed for one minute with each solution. Between each addition, the sensor was washed with PBS.

FIG. 5E shows a bar graph based off the selectivity study, in accordance with various embodiments of the present disclosure. Solutions with βHB present show a higher current versus those without βHB present.

FIG. 6A illustrates a portable detection unit that is connected to a Keto-sensor, in accordance with various embodiments of the present disclosure. This portable detection unit has a Bluetooth unit to send data to a tablet.

FIG. 6B shows an SEM image of pristine carbon on a screen-printed electrode, in accordance with various embodiments of the present disclosure.

FIG. 7 shows a dose-dependent plot for a representative enzymatic nanosensor, in accordance with various embodiments of the present disclosure.

FIGS. 8A-8D show a comparative analysis of machine learning techniques of Linear Regression (FIG. 8A), Decision Tree (FIG. 8B), Random Forest (FIG. 8C), and Support Vector Regression (FIG. 8D) for predicting βHB concentrations from nanosensor readings with model performance comparison and visualization of prediction accuracy across concentration ranges, in accordance with various embodiments of the present disclosure.

FIGS. 9A-9C demonstrate modeling complex relationships using polynomial regression analysis of βHB concentrations at degrees of n=2 (FIG. 9A), n=3 (FIG. 9B), and n=4 (FIG. 9C), in accordance with various embodiments of the present disclosure.

FIG. 10 is a schematic block diagram of an example of a processing or computing device, in accordance with various embodiments of the present disclosure.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### SUMMARY

In accordance with the purpose(s) of the disclosure, as embodied and broadly described herein, the disclosure, in one aspect, relates to systems comprising: a support; a potentiostat; a first electrode set into or layered on a surface of the support and in electrical communication with the potentiostat; a second electrode set into or layered on a surface of the support and in electrical communication with the first electrode and the potentiostat; and a third electrode set into or layered on a surface of the support and in electrical communication with the first electrode and the potentiostat; wherein the first electrode comprises at least one layer of graphene oxide coated onto a surface of the electrode and an enzyme layer coated onto a surface of the graphene oxide layer, wherein the enzyme layer comprises β-hydroxybutyrate dehydrogenase (βHBD), β-nicotinamide adenine (NADH), and an enzyme-stabilizing agent. Also disclosed herein are methods for measuring an analyte in a biological sample using the systems disclosed herein, comprising: obtaining a biological sample; bringing the biological sample into contact with the first electrode, second electrode, and third electrode of the system; and measuring at least one of: a) an open circuit potential between the first electrode and the third electrode; or b) a current flow between the first electrode and the second electrode.

Also disclosed are apparatuses comprising: a support; a first electrode set into or layered on a surface of the support; a second electrode set into or layered on a surface of the support; and a third electrode set into or layered on a surface of the support; wherein the first electrode comprises at least one layer of graphene oxide coated onto a surface of the electrode and an enzyme layer coated onto a surface of the graphene oxide layer, wherein the enzyme layer comprises β-hydroxybutyrate dehydrogenase (βHBD), β-nicotinamide adenine (NADH), and an enzyme-stabilizing agent.

Also disclosed herein are methods for fabrication a sensor, comprising: applying a first solution comprising graphene oxide nanosheets to an electrode; drying the first solution and electrode, thereby forming a graphene oxide-coated electrode; applying a second solution comprising N-Ethyl-N′-(3-dimethylaminopropyl) carbodiimide (EDC) and N-hydroxysuccinimide (NHS) to the graphene oxide-coated electrode, thereby forming a functionalized electrode; and applying a third solution comprising an enzyme-stabilizing agent, β-hydroxybutyrate dehydrogenase (βHBD), and β-nicotinamide adenine (NADH) to the functionalized electrode.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described aspects are usable in all aspects of the disclosure taught herein. Furthermore, the individual features of the dependent claims, as well as all optional and preferred features and modifications of the described aspects are combinable and interchangeable with one another.

### DETAILED DESCRIPTION

Disclosed herein are various examples related to a sensor (e.g., biosensor) used for selective detection of an analyte in a given sample. Reference will now be made in detail to the description of the embodiments as illustrated in the drawings, wherein like reference numbers indicate like parts throughout the several views.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1 percent to about 5 percent” should be interpreted to include not only the explicitly recited concentration of about 0.1 weight percent to about 5 weight percent but also include individual concentrations (e.g., 1 percent, 2 percent, 3 percent, and 4 percent) and the sub-ranges (e.g., 0.5 percent, 1.1 percent, 2.2 percent, 3.3 percent, and 4.4 percent) within the indicated range. The term “about” can include traditional rounding according to significant figures of the numerical value. In addition, the phrase “about ‘x’ to ‘y’” includes “about ‘x’ to about ‘y’”.

Furthermore, the terms “about”, “approximate”, “at or about”, and “substantially” as used herein mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Although specific terms are employed herein, they are used in a generic and descriptive sense only and not for purposes of limitation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

### A. DEFINITIONS

The terms “subject”, “individual”, or “patient” as used herein are used interchangeably and refer to an animal preferably a warm-blooded animal such as a mammal. Mammal includes without limitation any members of the Mammalia. A mammal, as a subject or patient in the present disclosure, can be from the order of Primates, Carnivora, Proboscidea, Perissodactyla, Artiodactyla, Rodentia, and Lagomorpha. In a particular embodiment, the mammal is a member of the family Bovidae, such as a cow. In other embodiments, animals can be treated; the animals can be vertebrates, including both birds and mammals. In aspects of the disclosure, the terms include domestic animals bred for food or as pets, including equines, bovines, sheep, poultry, fish, porcines, canines, felines, and zoo animals, goats, apes (e.g. gorilla or chimpanzee), and rodents such as rats and mice.

The term “limit of detection” or “LoD” as used herein refers to the lowest actual concentration of an analyte in a measured sample that can be consistently detected about greater than or equal to 95% of the time. Examples for calculating LoD can be found in the Examples.

### B. DISCUSSION

Herein is introduced a low-cost and highly sensitive electrochemical biosensor that can be used to detect a target analyte in a biofluid (serum, milk, urine, ruminal fluid, and the like) in a subject. In one aspect, the subject is livestock, such as dairy cows. In another aspect, the sensor is used to detect beta-hydroxybutyrate (βHB) to identify ketosis (i.e., a Keto-sensor). The sensor can be fabricated of relatively low-cost materials, have a good analytical sensitivity, and be configured for on-site testing and use.

Nanostructures with two-dimensional geometries can be used to construct biosensors to track biomarkers in biological and other media. This can allow surface functionalization to hold enzymes or antibodies due to their high surface area, and also enhance electron transport properties, resulting in rapid detection of the device. Though 2D materials, specifically graphene nanosheets, provide abundant functional groups (—COOH) to bind with enzyme molecules (—NH₂) covalently via amidation reactions, the enzymes ideally need a high stability on sensor surface. Enzyme stabilization is an important factor to consider for commercial field testing and wearable sensors. This provides several advantages such as a) increased sensor-to-sensor reproducibility and sensor shelf-life, b) reduced bio-fouling, c) maintained sensor functionalities, and d) reduced tendency of the enzyme to unfold. Thus, stabilization of enzymes could provide a more practical sensor for continuous monitoring of biomarkers to support precision livestock farming.

Precision livestock farming (PLF) is important to monitor the health status of dairy herds. Dairy farmers employ several precision technologies to improve livestock health, milk yield, and the well-being of animals. In this regard, on-site sensing technologies not only identify early signs of illness but also avoid cost and time, actionable decision-making for regulation operations of dairy herds. One metabolic disease is subclinical ketosis (SCK) in early lactating cows. This is due to the intense demand for glucose and the mobilization of adipose tissue during a cow's transition period. Elevated circulated ketone bodies (acetone (Ac), acetoacetate (AcAc), and βHB) in bodily fluids such as milk and blood are a symptom of subclinical ketosis, but there is no visual indication. Dairy cattle are very susceptible to SCK in their initial lactation due to high energy demands and limited feed intake post-partum. If not treated, a ketotic cow will lose its appetite, have decreased production and reproductive performance, and become more susceptible to other diseases such as mastitis, displaced abomasum, and metritis. Prevention of clinical ketosis in cows is less costly than treatment and loss of production due to this disease. It is estimated that the prevalence of SCK is high (˜40-60%) compared the clinical ketosis (˜2-15%). The cost of SCK includes the treatment of the animal, loss of milk production, and delays in conception. Estimates of costs per case vary, ranging from $24 to $1030, and averaging $165 per case based on stochastic simulation modeling. Rapid detection of SCK at its earliest stages can help to improve economic benefits for dairy farmers and allow them to make better management decisions for their animals.

Sensing of ketosis in humans has shifted from monitoring blood glucose levels to blood ketone levels, which has the potential to be a biomarker for ketosis. The quantification of βHB is one standard for the diagnosis of SCK in dairy cows. A dairy cow with concentrations of blood or serum βHB greater than 1.0 mM (or 1.4 mM) is considered to have subclinical ketosis. Standard thresholds for subclinical ketosis are 1.2 mM for blood, 100 μM for milk, and 1.5 mM for urine. Traditional diagnostic tools for ketosis sensing include dipstick tests and other laboratory-based diagnostic tests. Dipstick tests involve the stimulation of urination on a stick of paper. The color change on the paper reflects the number of ketones present in the cow's urine. This cow-side test is simple but not very precise or accurate. Another common tool to detect ketosis involves sending samples of blood, urine, or milk from cows to a laboratory to be analyzed using an enzymatic test based on spectrophotometry. Such assay can be used to accurately measure ketone levels in both blood or urine. However, sample processing and shipping from herds to testing facilities introduces additional time and cost factors, another major obstacle to managing dairy herds. To address these challenges, on-site biosensing tools could be helpful in the management of cow health for their rapid and early diagnosis of subclinical ketosis. On-farm detection could enable faster decisions, prevent clinical ketosis, and improve the economic benefits and well-being of cattle. Previously developed biosensors had low sensitivities in the range of millimoles (i.e., 0.05 mM). Commercially available ketosis tests are exclusively made for human monitoring; thus, they have limited sensitivities (less than 85%) compared to regular laboratory tests. Furthermore, some commercial tools suffer from significant false-positive results. Thus, there is an unmet need of developing reliable and low-cost ketosis biosensors which can allow for the on-farm detection of subclinical ketosis in dairy herds.

Disclosed herein is a biosensor system including a support; a potentiostat; a first or working electrode, set into or layered on a surface of the support and in electrical communication with the potentiostat; a second or counter electrode, set into or layered on a surface of the support and in electrical communication with the first electrode and the potentiostat; and a third or reference electrode, set into or layered on a surface of the support and in electrical communication with the first electrode and the potentiostat. The biosensor system can have a limit of detection of about 0.10 nM to about 0.30 nm, about 0.15 nM to about 0.30 nM, about 0.20 nM to about 0.30 nM, about 0.10 nM to about 0.25 nM, or about 0.10 nM to about 0.20 nM for a target analyte (e.g., βHB), optionally present in a biofluid (e.g., bovine biofluid). Also disclosed herein is a biosensor including a support; a first electrode set into or layered on a surface of the support; a second electrode set into or layered on a surface of the support; and a third electrode set into or layered on a surface of the support.

The first electrode can include at least one layer of graphene oxide coated onto a surface of the electrode and an enzyme layer comprising β-hydroxybutyrate dehydrogenase (βHBD), β-nicotinamide adenine (NADH), and an enzyme-stabilizing agent (a stabilizer), where the enzyme layer is covalently bonded to the graphene oxide layer. In one aspect, the enzyme layer comprises NADH and βHBD in about a 1:1 weight ratio. The enzyme layer can be bonded to the graphene oxide layer via an amide linkage between the graphene oxide and NADH. Enzyme-stabilizing agents can include compounds such as glycerol, poly(glycidyl methacrylate-ran-oligo(ethylene glycol) methacrylate) [poly(GMA-ran-OEGMA)], a combination thereof, and the like.

The support can be a paper material or a plastic material, such as polypropylene or polyethylene terephthalate. The electrode can be set into or placed onto the support. The electrode can be formed by applying an electrode-forming material to the support, such as by dip-coating. Any one of the electrodes can be a carbon-based electrode, including carbon in the form of graphite, carbon black, activated carbon, carbon nanotube, carbon nanofiber, graphene, a combination thereof, or the like; an MXene-based electrode, including MXene nanosheets; an MoS₂-based electrode, including MOS₂ nanosheets; or any combination thereof. MXenes are two-dimensional materials comprising carbides and nitrides of various transition metals, where atomically thin layers comprising transition metals are interleaved with atomically thin layers comprising carbon and/or nitrogen. The electrodes can further comprise nanoparticles, such as gold nanoparticles and/or metal oxide (e.g., ZnO, NiO) nanoparticles. In one aspect, the electrodes are comprised of the same materials. In another aspect, the electrodes can be comprised of at least one different material or completely different materials in comparison to one another. In one aspect, the electrodes are screen-printed electrodes.

In one aspect, the sensor disclosed herein includes a screen-printed electrode which is modified with graphene nanosheets to allow covalent functionalization aided by N-Ethyl-N′-(3-dimethylaminopropyl) carbodiimide (EDC)-N-hydroxysuccinimide (NHS) chemistry of enzymes. In one aspect, two enzymes are utilized (βHBD and NADH) and stabilized with a glycerol treatment before their immobilization. These enzymes on the sensor surface may allow for electrocatalytic reactions and generate electrons which can be measured via a graphene integrated SPE current collector. Enzyme stabilization improves sensor stability and selectivity. This sensor can be compared to control sensors, including a screen-printed sensor (S-sensor) and a screen-printed sensor with graphene nanosheets (G-sensor) and no enzymes. In one aspect, the sensor disclosed herein can detect βHB within about a minute and is sensitive to a nanomolar (e.g., about 0.1 nM to about 0.3 nM) concentration of βHB. This sensor also demonstrates the ability of continuous monitoring of βHB concentration.

Other Embodiments: In another aspect, a small, contained circuit and chassis system are constructed for the sensor. The circuit includes a voltmeter from the sensor, a microcontroller, and a Bluetooth component to receive the signal from the sensor, translate it based on calibration data and, optionally, connect it to a smartphone for data visualization. This allows for mobile testing setup that a person can use by hand. A continuous monitoring system built into farm equipment can also be constructed. Being able to connect to a smartphone can make the sensor more appealing to mass consumer markets. In another embodiment, the sensor has applications in preventative medicine.

Also disclosed herein is a method for fabricating a biosensor or sensor of the present disclosure, including applying a first solution comprising graphene oxide nanosheets to an electrode; drying the first solution and electrode, thereby forming a graphene oxide-coated electrode; applying a second solution comprising EDC and NHS to the graphene oxide-coated electrode, thereby forming a functionalized electrode; and applying a third solution comprising an enzyme-stabilizing agent, βHBD, and NADH to the functionalized electrode. In one aspect, the EDC and NHS can be present in the second solution in a weight ratio (EDC: NHS) ranging from about 1:5 to about 5:1, about 1:4 to about 4:1, about 1:3 to about 3:1, about 1:2 to about 2:1, about 1:2 to about 4:1, about 1:2 to about 3:1, about 1:4 to about 3:1, about 1:4 to about 2:1, about 1:1 to about 4:1, about 1:4 to about 1:1, or a weight ratio of about 1:1. In another aspect, the βHBD and NADH are present in the third solution in a weight ratio (βHBD: NADH) ranging from about 1:2 to 2:1, about 1:1 to about 2:1, about 1:2 to about 1:1, or a weight ratio of about 1:1. In another aspect, the third solution comprises the enzyme-stabilizing agent in an amount ranging from about 5% to about 50%, about 5% to about 40%, about 5% to about 30%, about 5% to about 20%, about 5% to about 10%, about 15% to about 50%, about 25% to about 50%, or about 35% to about 50% by volume. The first solution can be applied to the graphene oxide-coated electrode multiple times prior to applying the second solution, with an optional drying step between each application. For example, the first solution can be applied to the graphene coated electrode at least twice, at least three times, or at least for times. As another example, the first solution can be applied to the graphene coated electrode from one to four times. In another aspect, the graphene oxide-coated electrode can be washed prior to applying the third solution. The electrode can be washed using a solvent or buffer solution such as water or phosphate-buffered saline.

Also disclosed herein is a method for measuring an analyte (e.g., βHB) in a biological sample (e.g., bovine biofluid) using any biosensor system as disclosed herein, comprising: obtaining a biological sample; bringing the biological sample into contact with the first electrode, second electrode, and third electrode of the biosensor system; and measuring at least one of: a) an open circuit potential between the first electrode and the third electrode; or b) a current flow between the first electrode and the second electrode. The method can further comprising applying the measured current flow to a trained machine learning map, thereby generating a concentration of an analyte in the biological sample. The machine learning map can be generated by a machine learning model that has been trained based on a known analyte concentration and a known current flow. The machine learning model can be a machine learning regression model, such as a linear regression model, a decision tree regression model, a polynomial regression model, and a random forest regression model.

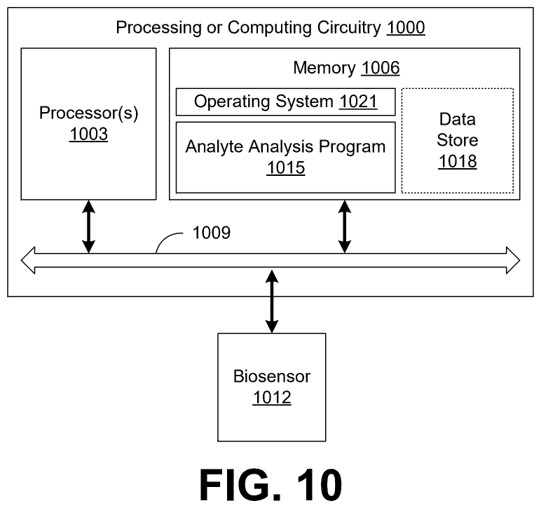

With reference to FIG. 10, shown is a schematic block diagram illustrating an example of processing or computing circuitry 1000. In some embodiments, among others, the processing or computing circuitry 1000 may include a processing or computing device such as, e.g., a smartphone, tablet, computer, etc. As illustrated in FIG. 10, the processing or computing circuitry 1000 can include, for example, a processor 1003 and a memory 1006, which can be coupled to a local interface 1009 comprising, for example, a data bus with an accompanying address/control bus or other bus structure as can be appreciated. To this end, the processing or computing circuitry 1000 may comprise, for example, at least one server computer or like device, which can be utilized in a cloud-based environment. In some embodiments, the processing or computing circuitry 1000 can include one or more network interfaces that may comprise, for example, a wireless transmitter, a wireless transceiver, and a wireless receiver. The network interface can communicate to a remote computing device using, e.g., a Bluetooth protocol or other wireless protocol.

In some embodiments, the processing or computing circuitry 1000 can include one or more network/communication interfaces. The network/communication interfaces may comprise, for example, a wireless transmitter, a wireless transceiver, and/or a wireless receiver. As discussed above, the network interface can communicate to a remote computing device using a Bluetooth, WiFi, or other appropriate wireless protocol. As one skilled in the art can appreciate, other wireless protocols may be used in the various embodiments of the present disclosure. In addition, the processing or computing circuitry 1000 can be in communication with a biosensor 1012 such as, e.g., a system or apparatus as disclosed herein. In some implementations, the biosensor 1012 can be coupled to the processing or computing circuitry 1000 and can interface through the locate interface 1009.

Stored in the memory 1006 can be both data and several components that are executable by the processor 1003. In particular, stored in the memory 1006 and executable by the processor 1003 can be an analyte analysis program 1015 and potentially other application program(s). Also stored in the memory 1006 may be a data store 1018 and other data. In addition, an operating system 1021 may be stored in the memory 1006 and executable by the processor 1003. The memory is defined herein as including both volatile and nonvolatile memory and data storage components. Volatile components are those that do not retain data values upon loss of power. Nonvolatile components are those that retain data upon a loss of power. Thus, the memory 1006 may comprise, for example, random-access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, optical disc such as compact disc (CD) or digital versatile disc (DVD), magnetic tapes accessed via an appropriate tape drive, holographic storage, and/or other memory components, or a combination of any two or more of these memory components. In addition, the RAM may comprise, for example, static random-access memory (SRAM), dynamic random-access memory (DRAM), or magnetic random-access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1003 may represent multiple processors 1003 and/or multiple processor cores (e.g., of a graphics processing unit), and the memory 1006 may represent multiple memories 1006 that operate in parallel processing circuits, respectively. In such a case, the local interface 1009 may be an appropriate network that facilitates communication between any two of the multiple processors 1003, between any processor 1003 and any of the memories 1006, or between any two of the memories 1006, etc. The local interface 1009 may comprise additional systems designed to coordinate this communication, including, for example, performing calibrations. The processor 1003 may be of electrical or of some other available construction.

A number of software components can be stored in the memory 1006 and can be executable by the processor 1003. An executable program may be stored in any portion or component of the memory 1006. In this respect, the term executable refers to a program file that is in a form that can ultimately be run by the processor 1003. Examples of executable programs may be, for example, a compiled program that can be translated into machine code in a format that can be loaded into a random access portion of the memory 1006 and run by the processor 1003, source code that may be expressed in proper format such as object code that is capable of being loaded into a random access portion of the memory 1006 and executed by the processor 1003, or source code that may be interpreted by another executable program to generate instructions in a random access portion of the memory 1006 to be executed by the processor 1003, etc. In particular, stored in the memory and executable by the processor can be a vehicle category classification program, an operating system and potentially other applications. Also stored in the memory may be a data store and other data. It is understood that there may be other applications that are stored in the memory and are executable by the processor as can be appreciated. Where any component discussed herein is implemented in the form of software, any one of a number of programming languages may be employed such as, for example, C, C++, C #, Objective C, Java®, JavaScript®, Perl, PHP, Visual Basic®, Python®, Ruby, Flash®, or other programming languages.

Although the analyte analysis program 1015 and other application program(s) or systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein. Also, any logic or application described herein, including the analyte analysis program 1015, that comprises software or code can be embodied in any non-transitory computer-readable medium for use by or in connection with an instruction execution system such as, for example, a processor 1003 in a computer system or other processing circuitry, device or system. In this sense, the logic may comprise, for example, statements including instructions and declarations that can be fetched from the computer-readable medium and executed by the instruction execution system. In the context of the present disclosure, a computer-readable medium can be any medium that can contain, store, or maintain the logic or application described herein for use by or in connection with the instruction execution system. In one aspect, the analyte analysis program 1015 can include machine learning.

The analyte analysis program 1015, which comprises an ordered listing of executable instructions for implementing logical functions, can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a computer-readable medium can be any means that can contain, store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection (electronic) having one or more wires, a portable computer diskette (magnetic), a random-access memory (RAM) (electronic), a read-only memory (ROM) (electronic), an erasable programmable read-only memory (EPROM or Flash memory) (electronic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory. In addition, the scope of the certain embodiments of the present disclosure includes embodying the functionality of the preferred embodiments of the present disclosure in logic embodied in hardware or software-configured mediums.

### C. ASPECTS

The following listing of exemplary aspects supports and is supported by the disclosure provided herein.
Aspect 1. A system comprising: a support; a potentiostat; a first electrode set into or layered on a surface of the support and in electrical communication with the potentiostat; a second electrode set into or layered on a surface of the support and in electrical communication with the first electrode and the potentiostat; and a third electrode set into or layered on a surface of the support and in electrical communication with the first electrode and the potentiostat; wherein the first electrode comprises at least one layer of graphene oxide coated onto a surface of the electrode and an enzyme layer coated onto a surface of the graphene oxide layer, wherein the enzyme layer comprises β-hydroxybutyrate dehydrogenase (βHBD), β-nicotinamide adenine (NADH), and an enzyme-stabilizing agent.
Aspect 2. The system of aspect 1, wherein the support is a paper support.
Aspect 3. The system of aspect 1 or aspect 2, wherein the first electrode is a carbon electrode.
Aspect 4. The system of any one of aspects 1-3, wherein the second electrode is a carbon electrode.
Aspect 5. The system of any one of aspects 1-4, wherein the third electrode is a carbon electrode.
Aspect 6. The system of any one of aspects 1-5, wherein the enzyme layer is covalently bonded to the graphene oxide layer via an amide linkage between the graphene oxide layer and NADH.
Aspect 7. The system of any one of aspects 1-6, wherein the enzyme-stabilizing agent is selected from glycerol, poly(GMA-ran-OEGMA), or a combination thereof.
Aspect 8. The system of any one of aspects 1-7, wherein the system has a limit of detection of about 0.10 nM to about 0.30 nM for a target analyte.
Aspect 9. The system of aspect 8, wherein the system has a limit of detection of about 0.10 nM to about 0.30 nM for a target analyte present in a biofluid.
Aspect 10. The system of aspect 9, wherein the biofluid is a bovine biofluid.
Aspect 11. The system of any one of aspects 8-10, wherein the target analyte is β-hydroxybutyrate.
Aspect 12. An apparatus comprising: a support; a first electrode set into or layered on a surface of the support; a second electrode set into or layered on a surface of the support; and a third electrode set into or layered on a surface of the support; wherein the first electrode comprises at least one layer of graphene oxide coated onto a surface of the electrode and an enzyme layer coated onto a surface of the graphene oxide layer, wherein the enzyme layer comprises β-hydroxybutyrate dehydrogenase (βHBD), β-nicotinamide adenine (NADH), and an enzyme-stabilizing agent.
Aspect 13. The apparatus of aspect 12, wherein the support is a paper support.
Aspect 14. The apparatus of aspect 12 or aspect 13, wherein the first electrode is a carbon electrode.
Aspect 15. The apparatus of any one of aspects 12-14, wherein the second electrode is a carbon electrode.
Aspect 16. The apparatus of any one of aspects 12-15, wherein the third electrode is a carbon electrode.
Aspect 17. The apparatus of any one of aspects 12-16, wherein the enzyme layer is covalently bonded to the graphene oxide layer via an amide linkage between the graphene oxide layer and NADH.
Aspect 18. The apparatus of any one of aspects 12-17, wherein the enzyme-stabilizing agent is selected from glycerol, poly(GMA-ran-OEGMA), or a combination thereof.
Aspect 19. A method for fabricating a sensor, comprising: applying a first solution comprising graphene oxide nanosheets to an electrode; drying the first solution and electrode, thereby forming a graphene oxide-coated electrode; applying a second solution comprising N-E thyl-N′-(3-dimethylaminopropyl) carbodiimide (EDC) and N-hydroxysuccinimide (NHS) to the graphene oxide-coated electrode, thereby forming a functionalized electrode; and applying a third solution comprising an enzyme-stabilizing agent, β-hydroxybutyrate dehydrogenase (βHBD), and β-nicotinamide adenine (NADH) to the functionalized electrode.
Aspect 20. The method of aspect 19, wherein the EDC and NHS are present in the second solution in a weight ratio ranging from about 1:4 to about 4:1.
Aspect 21. The method of aspect 19 or aspect 20, wherein the βHBD and NADH are present in the third solution in a weight ratio ranging from about 1:2 to about 2:1.
Aspect 22. The method of any one of aspects 19-21, wherein the third solution comprises from about 5% to about 50% by volume of the enzyme-stabilizing agent.
Aspect 23. The method of any one of aspects 19-22, wherein the first solution is applied to the graphene oxide-coated electrode at least one additional time prior to applying the second solution.
Aspect 24. The method of any one of aspects 19-23, wherein the graphene oxide-coated electrode is washed prior to applying the third solution.
Aspect 25. The method of any one of aspects 19-24, wherein the electrode is a carbon electrode.
Aspect 26. The method of any one of aspects 19-25, wherein the enzyme-stabilizing agent is selected from glycerol, poly(GMA-ran-OEGMA), or a combination thereof.
Aspect 27. A method for measuring an analyte in a biological sample using the system of any one of aspects 1-11, comprising: obtaining a biological sample; bringing the biological sample into contact with the first electrode, second electrode, and third electrode of the system; and measuring at least one of: a) an open circuit potential between the first electrode and the third electrode; or b) a current flow between the first electrode and the second electrode.
Aspect 28. The method of aspect 27, further comprising applying the measured current flow to a trained machine learning map, thereby generating a concentration of an analyte in the biological sample; wherein the machine learning map is generated by a machine learning model trained based on a known analyte concentration and a known current flow.
Aspect 29. The method of aspect 28, wherein the machine learning model is a machine learning regression model.
Aspect 30. The method of any one of aspects 27-30, wherein the biological sample comprises a bovine biofluid.
Aspect 31. The method of any one of aspects 27-31, wherein the analyte is β-hydroxybutyrate.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Ketosis Sensor

Disclosed herein is an example embodiment of the biosensor. The biosensor discussed herein (also referred to as a Keto-sensor) shows promising results as it can detect both the clinical and subclinical ketosis in the serum of dairy cows with a response time of less than a minute. Detecting βHB at such a low concentration (0.01 μM) can allow farmers to monitor the changes in their cows' bodies before any problems may arise, which can allow them to detect metabolic disease in its early stage. Additionally, the fast response time is ideal for field use of this sensor. This Keto-sensor shows promise in the lab setting displaying differences between samples spiked and not spiked without βHB. Providing farmers with fast and accurate data on their crops, land, or animals is especially important to implementing designs of biosensors for field use.

Materials and Methods. β-hydroxybutyrate dehydrogenase (βHBD), β-nicotinamide adenine (NADH), N-E thyl-N′-(3-dimethylaminopropyl) carbodiimide (EDC), N-hydroxysuccinimide (NHS), glycerol and fetal bovine serum were purchased from Sigma Aldrich, MO, USA. High quality single-layered graphene oxide nanosheets were bought from ACS Material, CA, USA. The highly-dispersed graphene solution of 0.5 mg/ml concentration was prepared in deionized (DI) water. According to the manufacturer, these 1-5 atomic layer graphene nanosheets were produced via thermal exfoliation followed by hydrogen reduction. The size of these nanosheets is varied from 0.5 μm to 5 μm having conductivity of 500˜700 S/m and the BET surface area is 650˜750 m2/g. The NHS stock solution was made by adding 28.7 mg NHS to 5 mL of phosphate buffered saline (PBS, 0.05 M solution) while EDC stock solution was made by adding 0.177 mL EDC to 4.82 mL PBS (0.2 M solution). The stock solution of βHBD was prepared by adding 1 mL PBS to the 25-unit bottle of βHBD. NADH stock solution was made by adding 1 mL PBS to the 25 mg powder of NADH. Deionized water, ACS reagent grade, ASTM Type I (Lab Chem, PA) having a resistance of 18.2 MΩ was also used to make buffer, sensing solution, spiking tests etc. A stock solution of 25 mM βHB purchased from Sigma Aldrich, MO, was created by the addition of 25 mg of βHB to 20 mL of phosphate buffer saline (PBS) solution. The PBS solution was prepared by adding 6 grams of potassium phosphate monobasic in 250 ml (0.2 M solution) of deionized water and 7.1 grams of sodium phosphate dibasic anhydrous added into 250 ml of deionized water (0.2 M solution), then 19 mL of the monobasic solution was added to 81 mL of the dibasic solution to reach a pH of 7.4. Next 100 ml of deionized water was added to the PBS solution and 1800 mg (0.9%) of NaCl (Fisher Chemical, MA), 329.26 mg of potassium ferricyanide (III) (Sigma-Aldrich, MO), and 422.39 mg of potassium hexacyanoferrate (II) trihydrate (Thermo Fisher Scientific, MA) was added and mixed until dissolved. This provides an equimolar concentration (5 M) of ferro/ferricyanide electrolyte probe to conduct experiments.

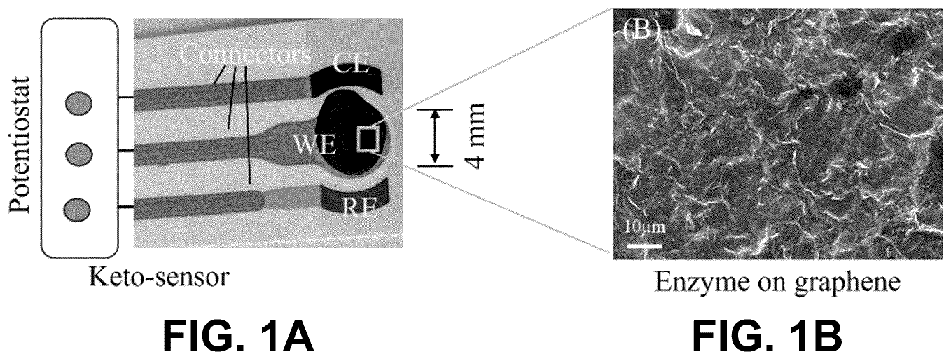

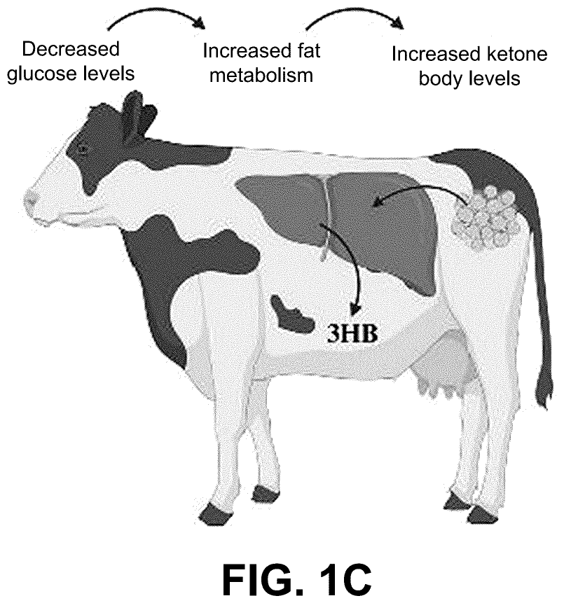

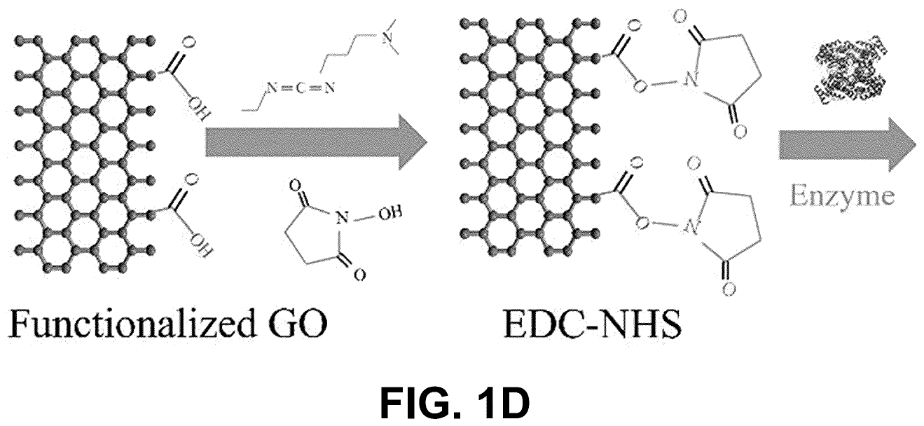

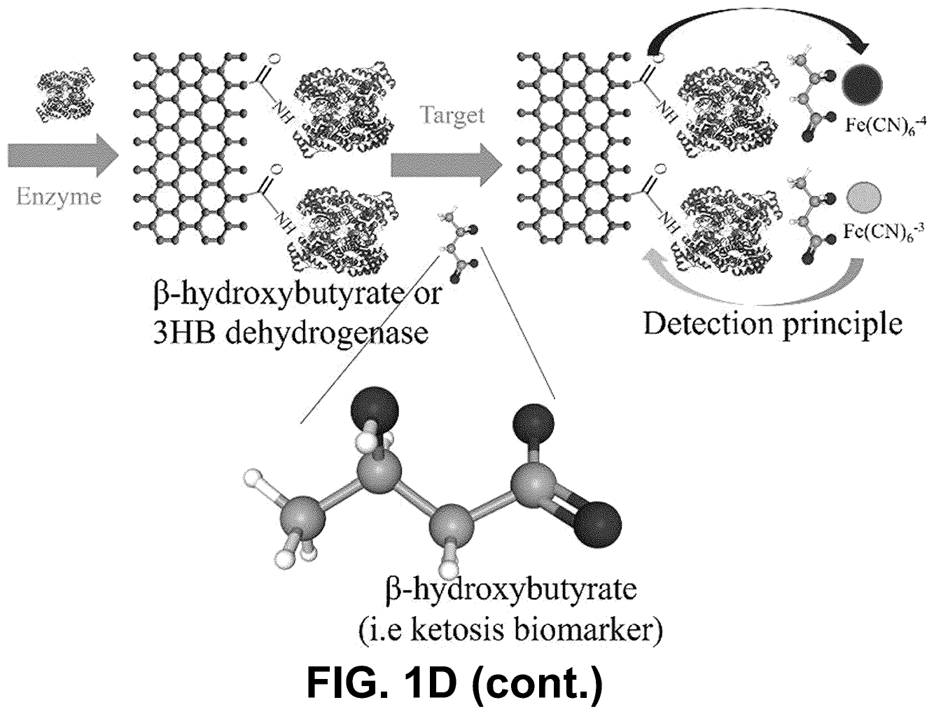

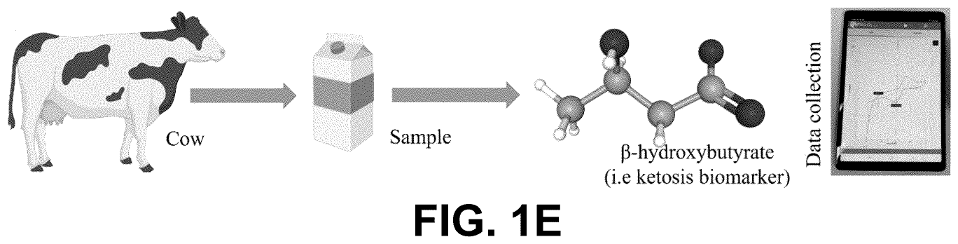

To build the sensors, several low-cost screen-printed carbon electrodes were obtained from BASi, Inc., IN. USA. This commercial electrode was chosen to avoid clean-room fabrication, reduce the device cost, and provide a high reproducibility of the sensor. Further, these sensors can easily be interfaced with a potentiostat readout for collections via Bluetooth. One sensor was inserted into a commercial readout (EmStat blue) to conduct experiments. Such Bluetooth-enabled potentiostat further connect via Bluetooth to a computer or tablet to collect data. Below are descriptions of the different experiments conducted using the ketosis electrochemical biosensor (Keto-sensor). The software used to collect data from the potentiostat was PSTrace (Palm Sens, Netherlands) and data were exported to Origin. Inc (OriginLab, MA) to create graphs. Additionally, BioRender was used to create the schematics in FIG. 1.

Device Fabrication. In one aspect, the keto-sensor comprises counter, working, and reference electrodes (FIG. 1). These electrodes are screen-printed onto a paper substrate. The working electrode (WE) can be modified. A photo of a keto-sensor is shown in FIG. 1A. A solution containing highly dispersed 2D graphene nanosheets was pipetted onto the WE and dried for one hour at 80° C. This procedure was done twice for a uniform surface of graphene. In this process, a thin layer of graphene nanosheets was layered due the non-covalent π-π interactions of graphene and carbon. Next, EDC and NHS solutions were applied at a one-to-one ratio to the working electrode and the sensor was placed in a humid chamber for four hours. After four hours, the electrode was washed with commercially available PBS (Gibco, MA), and the sensor was to functionalize with enzyme. This EDC-NHS treatment can activate the abundant-COOH groups on the graphene modified Keto-sensor to bind with proteins.

The step of sensor functionalization is shown in FIG. 1D. For enzyme functionalization, an enzyme solution was prepared. The enzyme solution consists of one-part NADH and one part beta-hydroxybutyrate dehydrogenase that are mixed at 1:1 weight ratio. To stabilize the enzyme, we utilized 5% glycerol that was mixed into the entire volume of the enzyme solution. 20 μL of this solution was spread uniformly on the surface of graphene electrode. In this EDC-NHS chemistry, —COOH groups at graphene surface have allowed to bind —NH₂ of enzyme and formed a C—N covalent bond via an amidation reaction. In brief, the EDC reacted with the graphene —COOH groups and formed o-acylisourea which can immediately react with NHS of enzyme molecules resulting in NHS ester. This NHS ester further can react with amine of an enzyme to form a C—N covalent bond. The sensor was placed in a humid chamber for at least four hours but left no longer than twelve hours. After the incubation, the electrode was washed again with PBS, then the sensor was placed in a 4° C. refrigerator until use.

The setup of the biosensor is illustrated in FIG. 1A. The potentiostat connects the three connectors that lead to the CE, WE, and RE. On the working electrode are layers of graphene with enzyme, represented by the scanning electron microscopy (SEM) images in FIG. 1B. FIG. 1C outlines how dairy cattle start to become ketotic. The glucose levels for the cow will decrease after calving because of the negative energy balance caused by increased lactation and decreased food consumption. Fat metabolism will take place to provide energy and ketone bodies like βHB are produced as a byproduct. FIG. 1D outlines the functionalization of the graphene layers. Graphene oxide was added to the screen-printed working electrode, EDC-NHS coupling with the graphene was done as described above, the enzyme solution was added to the sensor, and finally, sensing was performed with this functionalized Keto-sensor. βHBD catalyzes the βHBD to acetoacetate and vice versa. The role of NADH in this reaction is to act as a reductant for the reaction creating beta-hydroxybutyrate and NAD+ acts as an oxidant when the reaction moves from beta-hydroxybutyrate to acetoacetate. The glycerol in the enzyme solution helps to keep stability over time as reported by other researchers. Lastly, FIG. 1E outlines how the collection of samples from dairy cows can be used to monitor beta-hydroxybutyrate. In addition to Keto-sensor, two more sensors were chosen as controls in this study. These control sensors are S-sensor (screen-printed electrode based sensor) and G-sensor (graphene modified screen-printed sensor) and these sensors do not contain specific enzymes, i.e., βHBD.

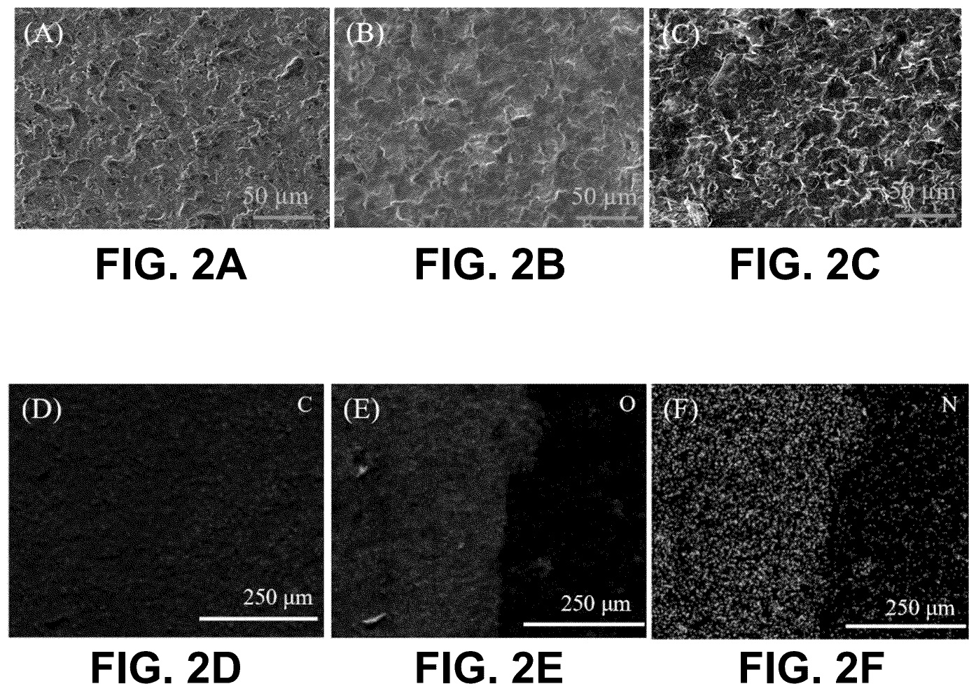

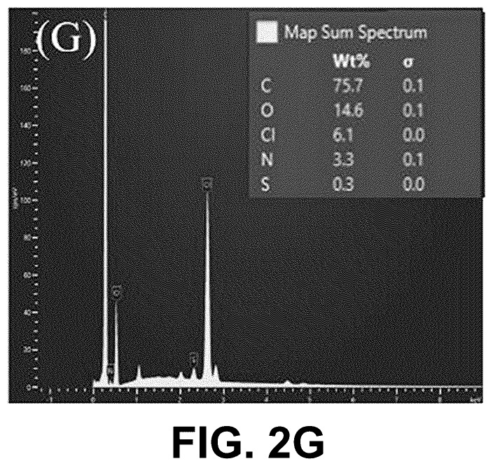

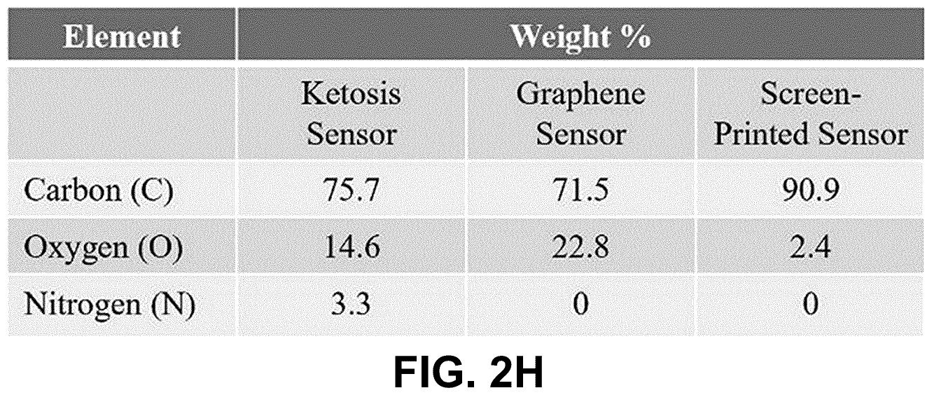

To investigate the surface morphologies of one embodiment of the sensors, scanning electron microscopy (SEM) imaging was conducted along with energy-dispersive X-ray spectroscopy (EDS). The SEM imaging was conducted at 500× for three different sensors: the screen-printed sensor without modification or S-sensor (FIG. 2A), the graphene sensor without enzyme or G-sensor (FIG. 2B), and the graphene nanosheets along with enzyme or Keto-sensor (FIG. 2C). The same sensors were used as SEM samples but coated with a thin layer of iridium. The screen-printed electrode shows the bare bulky carbon structure that is packed together with a non-uniform surface (FIG. 2A). This morphology is changed when spreading the nanosheets of graphene layers (FIG. 2B). These nanosheets are seen to be connected and formed a porous, thick layer. As expected, this porous layer is due to the π-π interactions among the nanosheets or carbon. Some nanosheets form wrinkles due to their stacking and folding. This nano-enabled sensor surface not only increases the area of surface reactions but also enhances the loading of enzymes. The morphology is further changed when we immobilize enzymes via EDC-NHS chemistry (FIG. 2C). The enzyme layer on graphene, however, is unclear to observe.

FIGS. 2D, 2E, and 2F display the mapping results from the EDS of the Keto-sensor to evaluate individual elements. The EDS spectrum of the Keto-sensor is displayed to show the presence of respective elements (FIG. 2G). The surface is completely covered in carbon (C˜90.9%) as the screen-printed electrode uses carbon (FIG. 2D). FIG. 2F shows the distribution of nitrogen (N) on the WE, which correlates to where the enzyme is attached to the graphene layer. The presence of N indicates the enzyme immobilization on the sensor surface. The addition of graphene nanosheets to both the Keto-sensor and G-sensor is likely the cause of the increase in the oxygen (O) weight percentage (wt %) of the working electrodes as compared to the S-sensor seen in FIG. 2H. N is present on its WE, but not on the working electrodes of the S-sensor and G-sensor due to the addition of β-hydroxybutyrate dehydrogenase to the Keto-sensor (FIG. 2H). A comparison table between the three different sensors is shown in FIG. 2H.

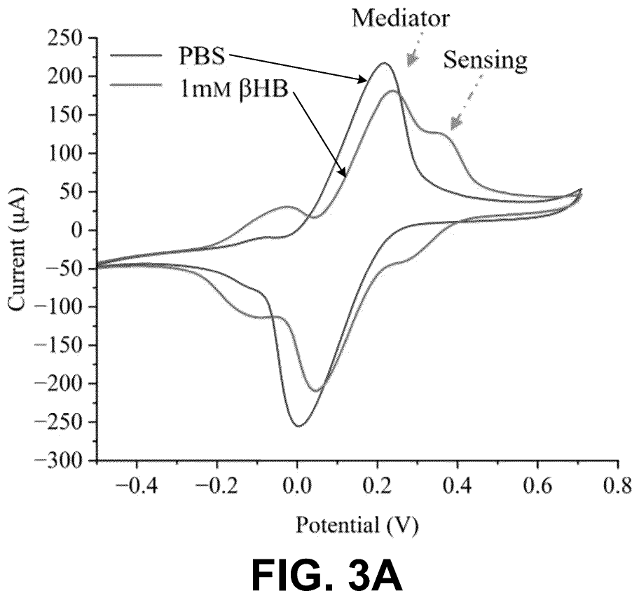

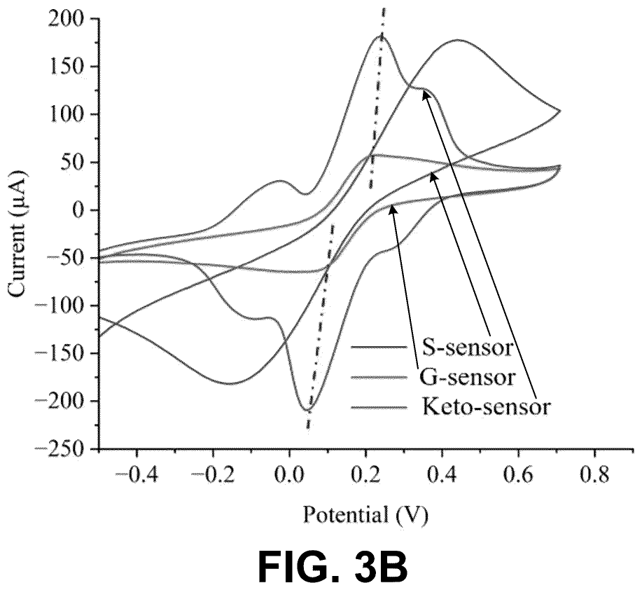

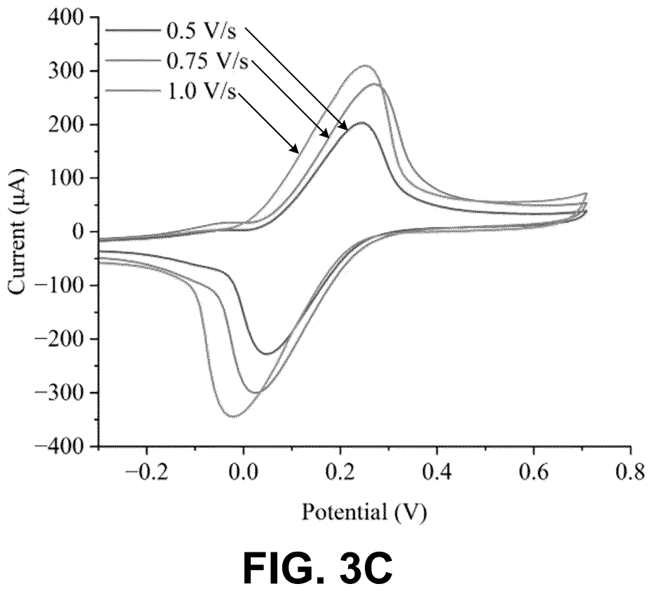

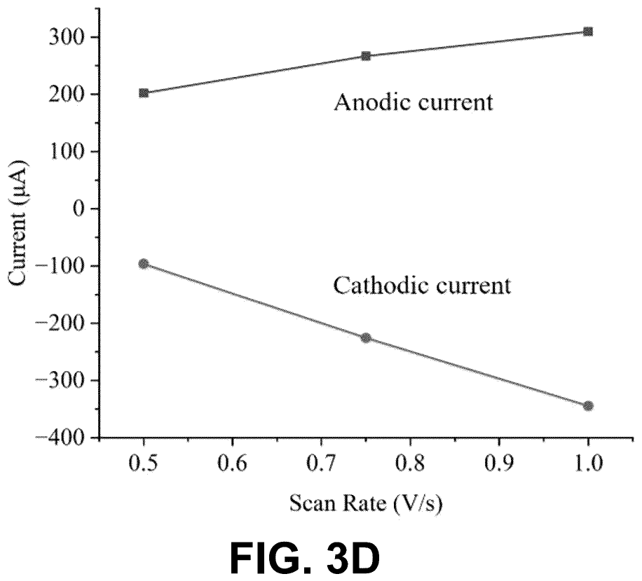

Electrochemical Sensing of Ketosis and Characterization. To investigate the redox property of the sensors, electrochemical studies were conducted of the Keto-sensor, S-sensor, and G-sensor. The CV measurements of all sensors were conducted using a PBS solution containing an equimolar concentration (5 mM) of a ferro/ferricyanide redox mediator. FIG. 3A shows the sensing of target molecules (βHB) via cyclic voltammetry (CV) using the Keto-sensor. It showed that both redox peaks (oxidation and reduction) are without βHB molecules (black graph), but no additional peaks. With βHB (1 mM), the Keto-sensor showed the same redox peaks in the CV graph (red graph), but the peak currents were found to be decreased and shifted towards right. A dominant oxidation peak appeared at 0.37 V with βHB concentration. This peak can be considered a sensing potential for the measurements of other βHB concentrations. This indicates that the Keto-sensor can detect the presence of βHB in buffer selectively. As Keto-sensor contains enzymes which catalyze the βHB and produces electrons at an oxidation potential of 0.37 V. At near 0 V, the Keto-sensor sensor showed another peak that may be due to the multiple materials that were used in the sensor construction. The CV tests used to show the comparison results among the S-sensor, G-sensor, and Keto-sensor while measuring βHB (1 mM) concentration are presented in FIG. 3B. The S-sensor has only carbon as a sensor's material and no enzyme. This sensor showed strong oxidation and reduction peaks in presence of βHB (1 mM). The sensing potential and current are found to be at ˜0.42 V and 150 μA. Once graphene nanosheets were added (G-sensor without enzyme), this sensing potential decreases to 0.3 V and current decreases (50 μA) significantly. This lower current is due to the available functional groups at the graphene surface that blocks the electron transfer from electrolyte to electrode. Overall, both G-sensor and S-sensor have different performance to detect βHB concentration. Though the current is lowered in G-sensor, the G-sensor configuration was used to add stabilized enzyme due to lower its lower sensing potential and enhance the selectivity of the sensor. Both the sensors have been further characterized to evaluate their sensing performances. As demonstrated in FIG. 3A, the Keto-sensor notably has multiple oxidation and reduction peaks versus the S-sensor and G-sensor. The addition of the enzyme on the Keto-sensor shows the creation of additional peaks, which show that the enzyme is reacting with the analyte causing the loss and gain of electrons, resulting in high selectivity. Ultimately, the Keto-sensor boosts the sensing signal more than three-times even if there is an enzyme coating on the sensor compared to G-sensor. This is due to β-hydroxybutyrate dehydrogenase which produces more electrons due to the enzymatic reactions during the detection of βHB concentration. Unlike Keto-sensor, the G-sensor and S-sensor did not show any other peaks in their respective CV graphs (FIG. 3B) during sensing of βHB concentration.

Scan rate study was performed using the Keto-sensor. For this study, a range of potential from −0.7 V to 0.7 V was applied with respect to RE. FIGS. 3C and 3D show the results of scan rate studies that were conducted using the Keto-sensor and tested scan rates of 0.5 V/s, 0.75 V/s, and 1.0 V/s. With the increase in scan rate, the oxidation peaks during the cyclic voltammetry increased, indicating the surface-controlled process of Keto-sensor. With a change in scan rate, there is a shift in the oxidation peak current to the right, and a shift in the reduction peak current to the left with increasing scan rate. The peak currents are found to be linearly proportional to the scan rate. With electrochemical characterizations, we have investigated the sensing performance of all sensors developed in this study. Though Keto-sensor provided a comparable sensing with S-sensor, it can be expected that Keto-sensor can provide more selectivity due to adding an enzyme layer. Lower sensing potential of Keto-sensor is another potential advantage compared to S-sensor.

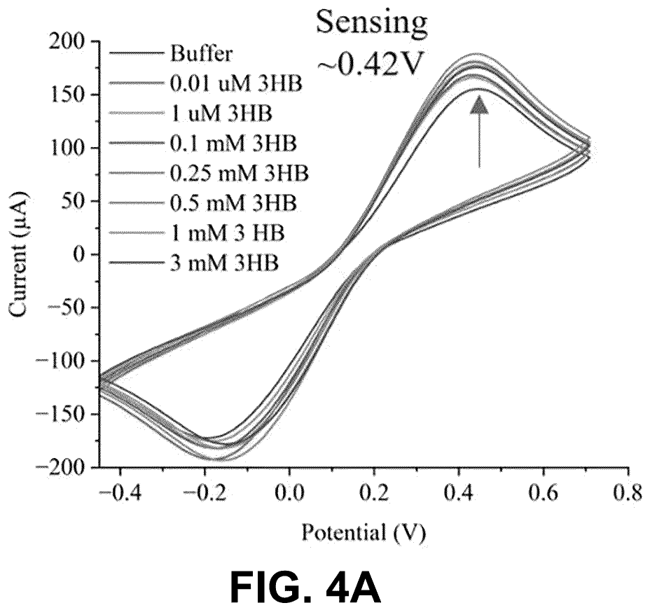

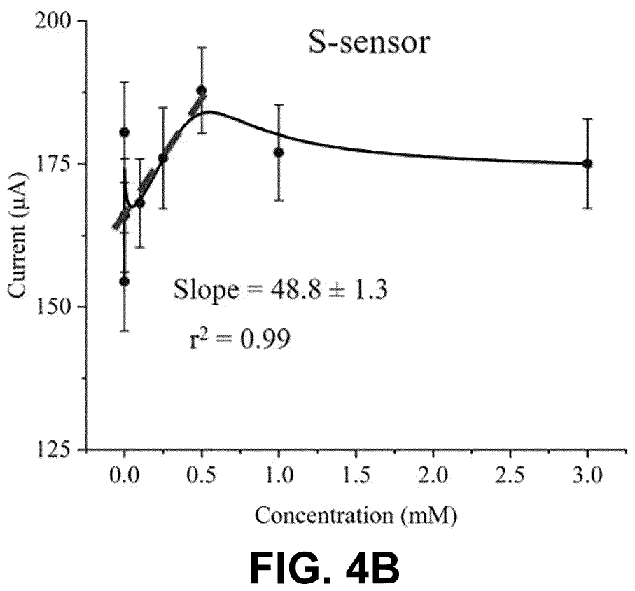

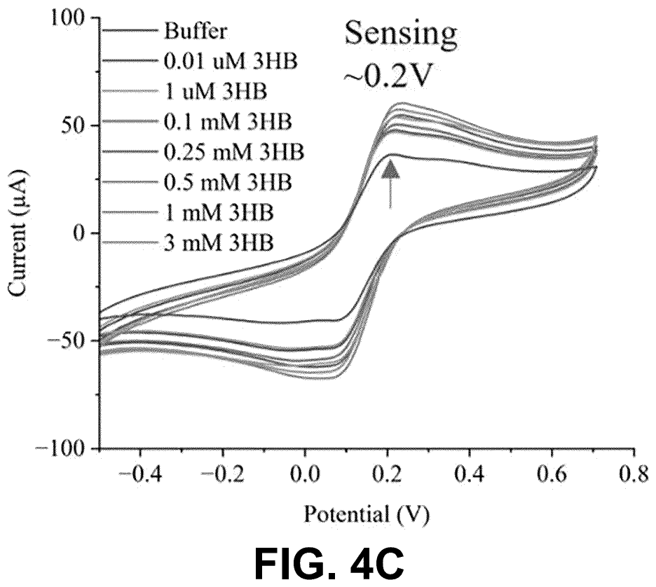

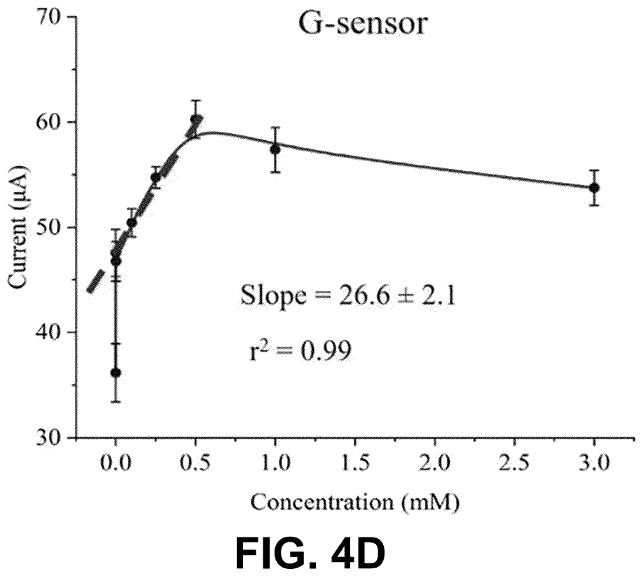

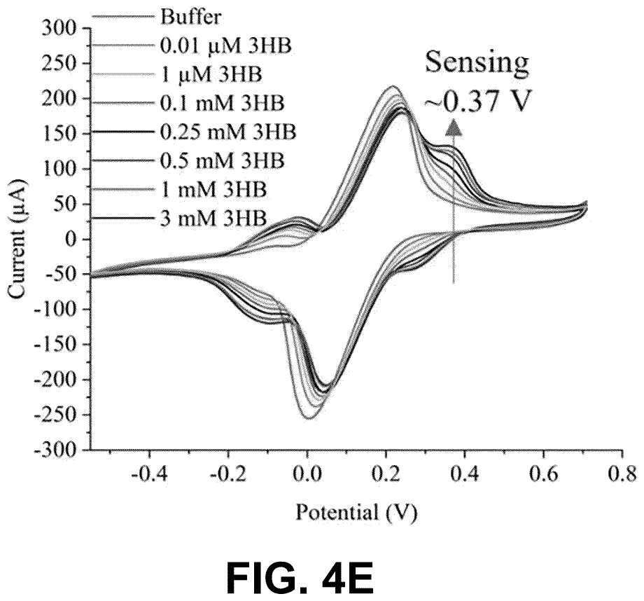

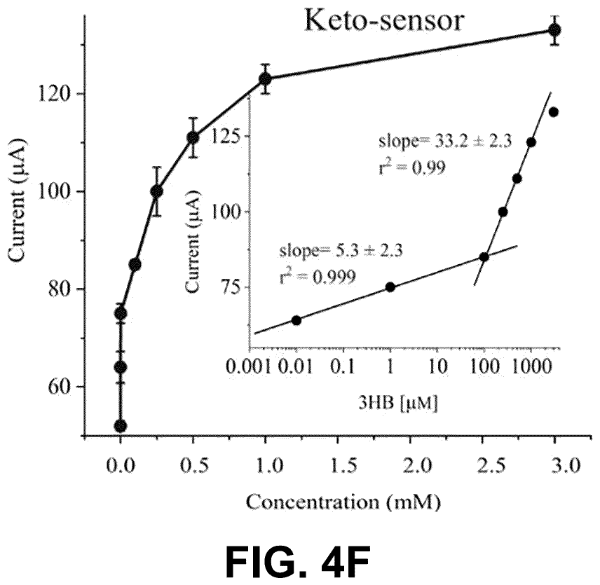

Ketosis (βHB) Sensing. To calibrate the sensors, both enzymatic and non-enzymatic sensing performances were conducted for all of the sensors. In non-enzymatic sensors, both S-sensor and G-sensor perform electrocatalytic reactions without any enzymes, while enzymatic Keto-sensor performs electrocatalytic reactions in the presence of enzymes. Carbon and graphene can act as electrocatalysts, which are useful materials to construct the non-enzymatic sensors. A CV test was adopted to calibrate all the sensors at a scan rate of 1 V/s. A number of dose-dependent standard solutions of βHB were prepared, ranging from 0.01 μM, 1.00 μM, 0.10 mM, 0.25 mM, 0.50 mM, 1.00 mM, and 3.00 mM in buffer. This can cover the physiological range of βHB (SCK˜ blood 1.2-2.9 mM and clinical ketosis ˜≥3.0 mM) in dairy cows. Testing samples were prepared using a PBS (50 mM, and pH˜7.4) solution containing an equimolar concentration (5 mM) of ferro/ferricyanide. This buffer without βHB was used to establish the sensor's baselines. FIG. 4 shows all sensing measurements for all sensors. For each concentration, three repeated measurements were taken with biologically independent solutions. Error bars were calculated by tacking the standard deviation of the three measurements.

βHB sensing results for the S-sensor are shown in FIG. 4A-B with dose-dependent concentrations. This was done using CV measurements (FIG. 4A). Initially, the S-sensor was exposed to a buffer solution to set the sensor baseline. Clear oxidation and reduction peaks were observed due to the presence of a ferro/ferricyanide mediator. The oxidation current is found to be 155 μA. Next, a minimum concentration of βHB (0.01 μM) was introduced to the S-sensor. The peak current was enhanced significantly compared to the sensor's baseline. This is due to the electrocatalysis of βHB molecules on the carbon surface. Then, the S-sensor was washed with buffer solution before we introduced the next target concentration. Further, the βHB concentration was increased from 0.1 μM to 3 mM and their signals were measured. The signal increases while increasing the concentration of βHB. The peak current is directly proportional to the dose-dependent concentration of βHB. The currents obtained from each CV graph were plotted against the βHB concentration (FIG. 4B). However, the S-sensor signal becomes saturated after 0.5 mM concentration of βHB. This is one of the major limitations of S-sensor that has been overcome by introducing a graphene layer along with a stabilized enzyme on the sensor surface. From the sensor calibration, the slop value was estimated as ˜48.8±1.3 μA.

Similarly, sensing measurements were conducted for the G-sensor in presence of all standard target concentrations of βHB (FIG. 4C-D). On adding a graphene layer, the oxidation peak potential is drastically reduced to 0.2 V. Furthermore, the peak current of the baseline's G-sensor is also reduced to 33 μA. The lesser baseline signal is due to the presence of defects and functional groups in graphene sheets. When a low concentration of βHB (0.01 μM) was introduced, the peak current of the G-sensor was increased notably. Further, upon increasing the concentration of βHB, the peak current was increased to a potential of 0.2 V. However, the peak current was found to stop increasing at a 0.5 mM concentration of βHB, after which the G-sensor showed a saturated results as like the S-sensor. For this sensor, the slop of the calibration is estimated as ˜26.6±2.6 μA (FIG. 4D). The limit of detections (LoDs) for both the S-sensor and G-sensor are estimated as 857.02 nM, and 545.56 nM, respectively. The detailed calculation of LoD is demonstrated in the Additional Information below. However, though the analytical sensitivities of both sensors are 0.01 μM, these sensors are not sensitive beyond 0.5 mM concentration of βHB.

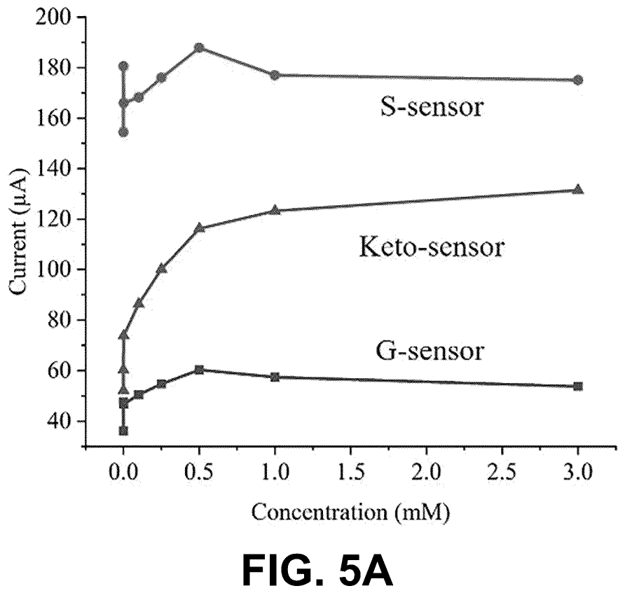

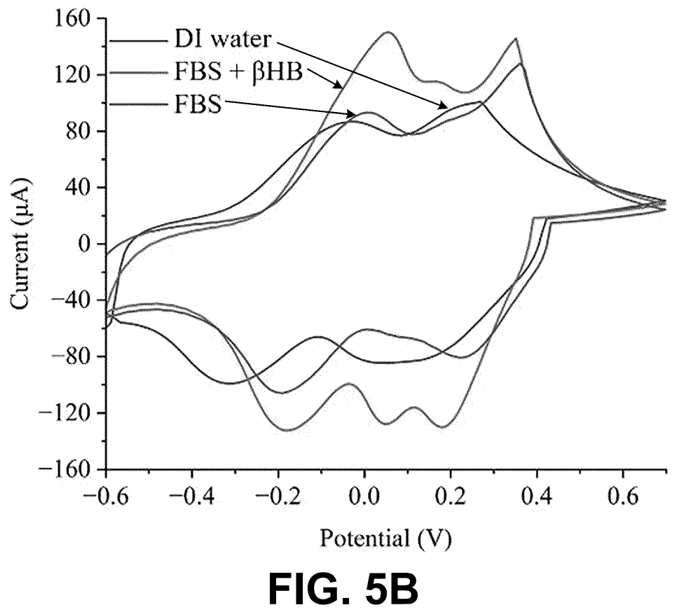

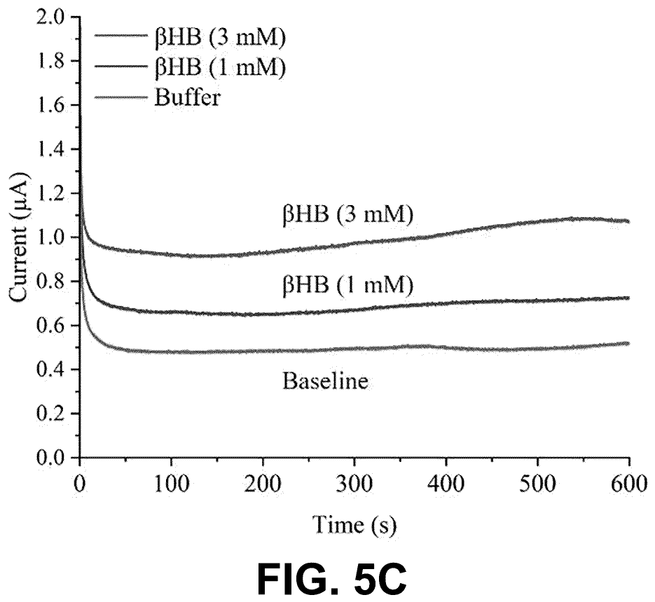

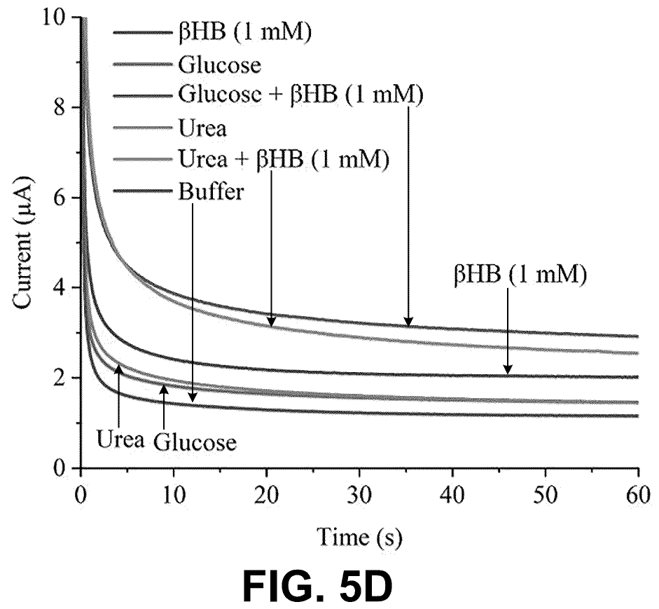

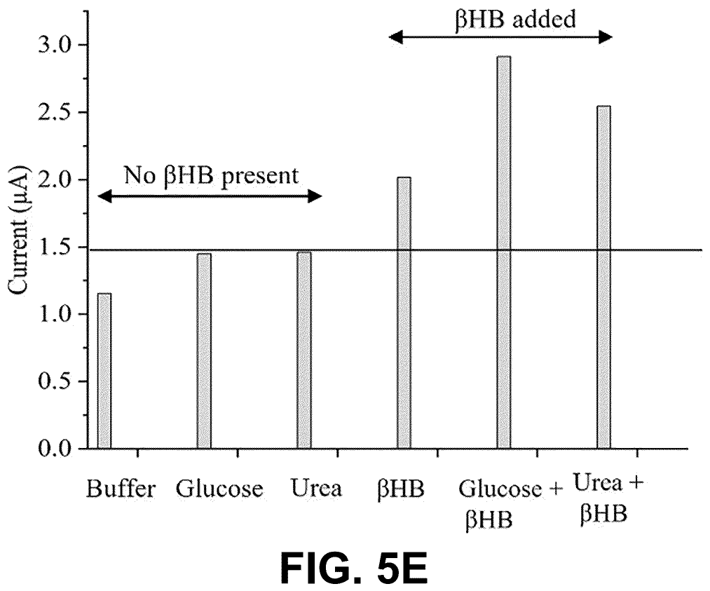

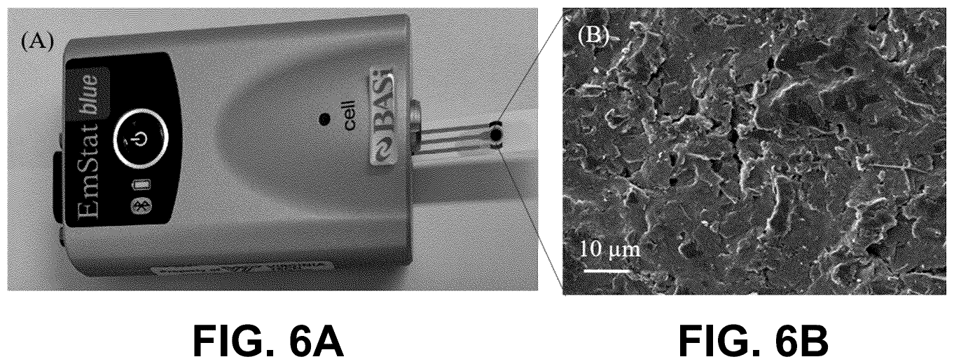

Sensing characterization of the Keto-sensor was performed using the same range of βHB concentration (FIG. 4E-F). At a specific potential (0.37V), the peak current increases while increasing the concentration of βHB concentration. Due to the enzyme layer in the Keto-sensor, a distinct peak sensing appeared at potential 0.37 V which is separated from the main redox peak in the CV graph (FIG. 4E). This feature of the Keto-sensor helps to detect a wider range of βHB concentration. In the Keto-sensor, the enzymatic reaction that takes place on the WE is beta-hydroxybutyrate and NADH taking part in an oxidation-reduction reaction. The beta-hydroxybutyrate is oxidized to make acetoacetate while the NADH is oxidized to create NAD+and provide the electron to reduce the acetoacetate back into beta-hydroxybutyrate. The reaction can be written as (R)-3-hydroxybutanoate+NAD+⁺↔acetoacetate+NADH+H⁺. This enzymatic reaction is responsible for increasing the electrochemical current during sensing of βHB. Additionally, on all sensors, the reaction K₄Fe(CN)₆↔K₃Fe(CN)₆ takes place on the working electrode. The Keto-sensor in this study has shown two linear ranges of detection. One is detection at low concentration from 0.01 μM to 100 μM with a slop value of 5.3±2.3 μA and the other is detection at concentrations of 100 μM to 3000 μM with a slop value of 33.3±2.3 μA. The LoD of this Keto-sensor is estimated to be at 0.24 nM (see the calculation in Additional Information below). This Keto-sensor has a high detection range compared to the S-sensor and G-sensor which may be due to adding an enzyme layer on the sensor surface. A comparison plot showing the sensing difference of all three sensors is illustrated in FIG. 5A. With the curves overlayed it is shown that the ketosis sensor has the best differentiation between different concentrations of βHB. With the S-sensor and G-sensor, the higher concentrations of βHB are harder to distinguish as sensors become saturated.

FIG. 5B shows results for spiked samples testing fetal bovine serum (FBS). The FBS was measured with and without βHB added. The Keto-sensor in FBS showed the same shape as CV graphs containing two dominant peaks with and without βHB and compared to measurements of FBS-only and DI water samples. FBS may pre-exist βHB, thus the sensor showed a peak current at ˜0.4V. When the FBS solution was spiked with a higher concentration (3 mM) of βHB, the peak current increased at the same potential, indicating the Keto-sensor can detect βHB in bovine serum.

FIG. 5C shows long-term and continuous measurement of βHB. For this, a chronoamperometry method was conducted in a buffer and different concentrations of βHB. The Keto-sensor was first set to a baseline that is without any target concentration. 50 μL of solution was pipetted onto the sensor and the current was measured continuously for 10 minutes. The Keto-sensor was tested with βHB concentrations of 1 mM and 3 mM and compared to the buffer solution. A potential of 0.37 V was applied to each sensor for the 10 minutes of measurement. With this experiment, the data shows that the Keto-sensor can detect concentrations of βHB that would be considered subclinical ketosis and below subclinical levels over extended periods of time. These results show promise for the possibility of continuous monitoring with these sensors. Farmers could use this device to monitor animal health before it reaches subclinical levels and make management decisions to prevent animal health from declining.

Table I compares a number of sensors developed for detecting βHB with the Keto-sensor created during this study. Many of the devices made with the goal of measuring βHB focus on use with human patients, however, this device and another device shown in Table I were made with the intended goal of on-farm use. Additionally, other devices tend to use amperometry for measuring, while this device and one other sensor used cyclic voltammetry measurements. The limit of detection for the Keto-sensor is 0.24 nanomolar, while the limit of detection for the G-sensor and S-sensor were 545.56 nanomolar and 857.02 nanomolar, respectively. The Keto-sensor has a much lower limit of detection than the other sensors (Table I). The Keto-sensor can detect both the clinical and subclinical ketosis in dairy cows as it covers detection from low to high levels of βHB (0.00001-0.1 mM and 0.25-3.0 mM). This capability shows the advantages of the Keto-sensor using graphene nanosheets with a stabilized enzyme onto the working electrode. Further, long-term and continuous measurement of βHB of this Keto-sensor is another important feature of the Keto-sensor compared to the reported literature.

TABLE I
Comparison table shows the sensor performance with other report literature.

| | | | Linear | | |
|---|---|---|---|---|---|
| Electrode | Biochemical | Sensing | Range & | Test | |
| materials | reactions | modalities | LOD | samples | Reference |
| Functionalized | βHB + NAD⁺ (3- | CV + | 0.00001-0.1 | Spiked | This work |
| graphene | HBDH)→NADH. | 370 mV | mM and | Bovine | |
| | Detect. NADH | | 0.25-3.0 | Serum | |
| | | | mM/0.24 nM | | |
| Graphene | βHB + NAD⁺ (3- | Amperometry + | 0.2-2.0 mM/— | Bovine | Veerapandian |
| | HBDH) → NADH. | 60 mV vs. | | Serum | et al., 2016 |
| | Detect. NADH | Ag/AgCl | | | |
| | with [Ru(bpy)₃]²⁺ | | | | |
| Iridium | βHB + NAD⁺ (3- | Amperometry + | 0-10 mM/— | Bovine | Fang et al., |
| functionalized | HBDH)→NADH | 200 mV vs. | | Serum | 2008 |
| carbon | Detect. NADH | Ag/AgCl | | | |
| Reduced | βHB + NAD⁺ (3- | Amperometry | 0.003-0.4 | Spiked | Martinez- |
| graphene | HBDH)→NADH | 0 mV vs. Ag | mM/0.001 mM | Human | Garcia et |
| | Detect. NADH | | | Serum | al., 2017 |
| | with THI | | | | |
| Carbon | βHB + NAD⁺ (3- | CV − | 0.01-0.1 | Human | Khorsand et |
| nanotube | HBDH)→NADH. | 150 mV vs. | mM/0.009 mM | Serum | al., 2013 |
| | Detect. NADH | Ag/AgCl | | | |

Selectivity Studies. The selectivity test of the Keto-sensor is shown in FIGS. 5D-5E. For the selectivity test, the most common target molecules available in serum samples of dairy cows were chosen. Glucose (Sigma-Aldrich, MO), urea (Fisher Chemical, MA), and βHB were used in selectivity testing. A glucose solution was made with the concentration of 2 mg/ml and a urea solution was made with the concentration of 2.5 mg/mL. Further, 1 mM βHB was added to the urea and glucose solutions to test for selectivity. First, the Keto-sensor was set to the baseline. On exposing glucose and urea, the Keto-sensor did not show a significant change. However, the Keto-sensor provided a sensing signal while the samples containing βHB were introduced. Adding glucose and urea into βHB concentration, the Keto-sensor showed a slight change in response current. However, their relative standard deviation (rsd) was estimated as ±9.1%. Such low rsd indicates that the selectivity is good and means the Keto-sensor is selective with being able to detect βHB in the presence of other compounds. FIG. 5D demonstrated the chronoamperometric graphs and FIG. 5E is a bar graph made from FIG. 5D to show the difference in current measured from each solution. This stabilized enzyme is responsible for such selectivity as this enzyme can have only biochemical reactions with βHB during detection.

Calculation of limit-of-detection (LoD). The limit-of-detection (LoD) was calculated for the S-sensor, G-sensor, and Keto-sensor. The limit-of-blank (LoB) was calculated by Equation 1.

$$LoB = Mean of signal ( blank sample ) + 1.645 \times ( SD of blank sample ) \qquad Equation 1$$

$${LoB}_{S - sensor} = 138.10 + 1.645 \times 7.75 = 150.85$$

$${LoB}_{G - sensor} = 33.48 + 1.645 \times 2.12 = 36.96$$

$${LoB}_{Keto - sensor} = 50.64 + 1.645 \times 7.61 = 63.15$$

Next, LoDs of the sensors were calculated by Equation 2.

$$LoD of the signal ( {Y}_{LoD} ) = LoB + 1.645 \times ( SD of target at low concentration ) \qquad Equation 2$$

$${Y}_{LoD} for S - sensor = 150.85 + 1.645 \times 5.70 = 160.23$$

$${Y}_{LoD} for G - sensor = 36.96 + 1.645 \times 2.10 = 40.40$$

$${Y}_{LoD} for Keto - sensor = 63.15 + 1.645 \times 3.87 = 69.51$$

Using the calibration curve of each sensor, the Y LoD for each sensor was calculated using Eq (3), where m is the slope of the calibration curve and c is the intercept.

$${Y}_{LoD} ( Ω ) = mX + c \qquad Equation 3$$

$${Y}_{LoD} ( Ω ) for the S - sensor = 48.8 \times Log [ X ] + 163.23$$

$${Y}_{LoD} ( Ω ) for the G - sensor = 26.6 \times Log [ X ] + 47.4$$

$${Y}_{LoD} ( Ω ) for the Keto - sensor = 6.52 \times Log [ X ] + 93.01$$

From Equations (2) and (3):

$$Log [ X ( nm ) ] for the S - sensor = ( 16 \; 0.23 - 163.23 ) / 48.8 = - 0.067$$

$${LoD}_{S - sensor} = 857.02 nM$$

$$Log [ X ( nm ) ] for the graphene sensor = ( 40.40 - 47 \text{.4} ) / 26.6 = - 0.263$$

$${LoD}_{G - sensor} = 545.56 nM$$

$$Log [ X ( nm ) ] for the ketosis sensor = ( 69.51 - 93.01 ) / 6.52 = - 3.604$$

$${LoD}_{Keto - sensor} = 0.24 nM$$

### 2. Machine Learning Assisted-Ketosis Sensor

Disclosed in this Example is a machine learning-assisted, relatively low-cost, sensitive electrochemical nanosensor for detected βHB to identify SCK in dairy cows. A machine learning approach has been applied to a calibration dataset to enhance the accuracy of predicting unknown concentrations of βHB. Briefly, the sensor or nanosensor features a screen-printed electrode modified with graphene nanosheets, enabling covalent functionalization through EDC and NHS chemistry. The sensor employs two enzymes, βHBD and NAD⁺, stabilized in glycerol prior to immobilization. Real milk samples were used to evaluate sensor performance. Several regression models were applied to the concentration versus sensor readout signal plot to evaluate the mean squared error (MSE) and co-efficient of determination (2), allowing for improved prediction of unknown βHB concentrations.

Traditional nanosensors that rely on linear relationships between concentration and sensor readings often fail to provide accurate predictions for unknown concentrations of target analytes. This is due to several factors, including non-linear sensor responses across varying concentrations, and matrix effects from other components in complex biofluids. Linear calibrations have a limited dynamic range and may not account for real sample variability. Additionally, variations in sensitivity caused by factors like temperature, bio-fouling effect, and pH can introduce inaccuracies. These challenges highlight the opportunity for using more advanced modeling approaches, such as non-linear regression or machine learning, to potentially improve prediction accuracy in complex environments.

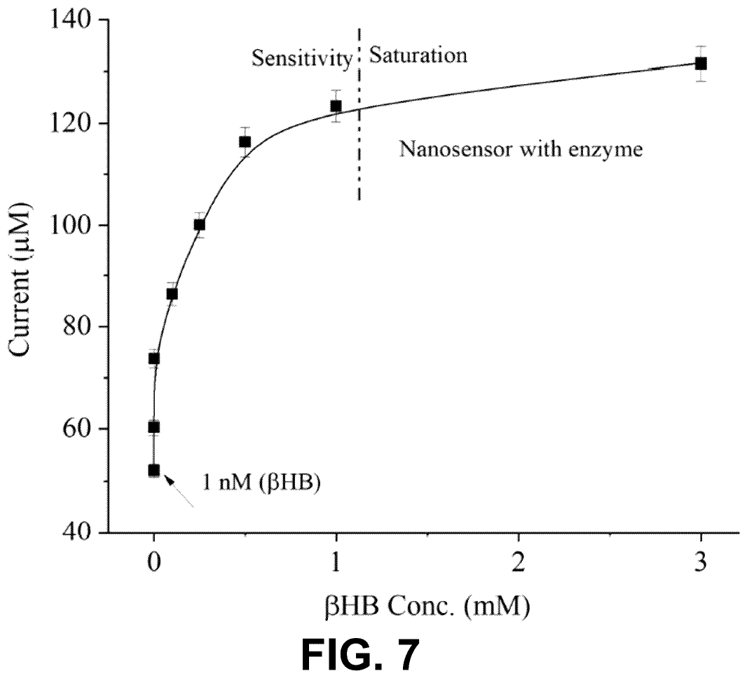

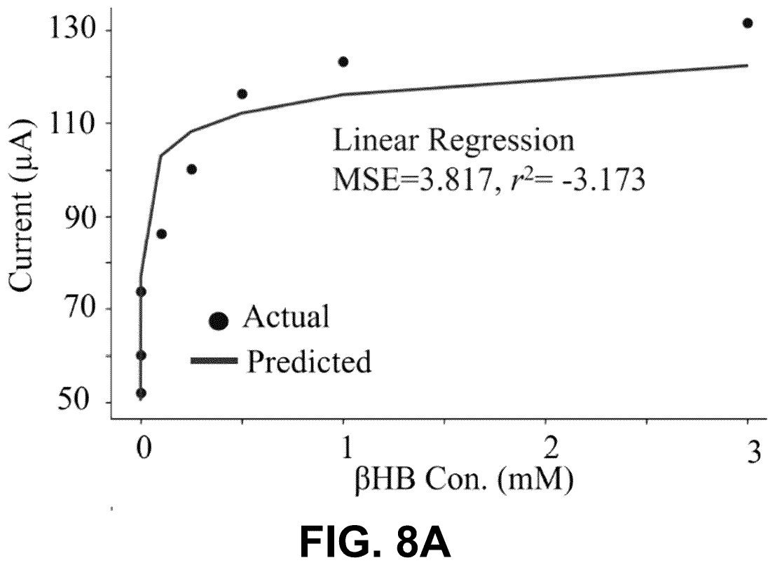

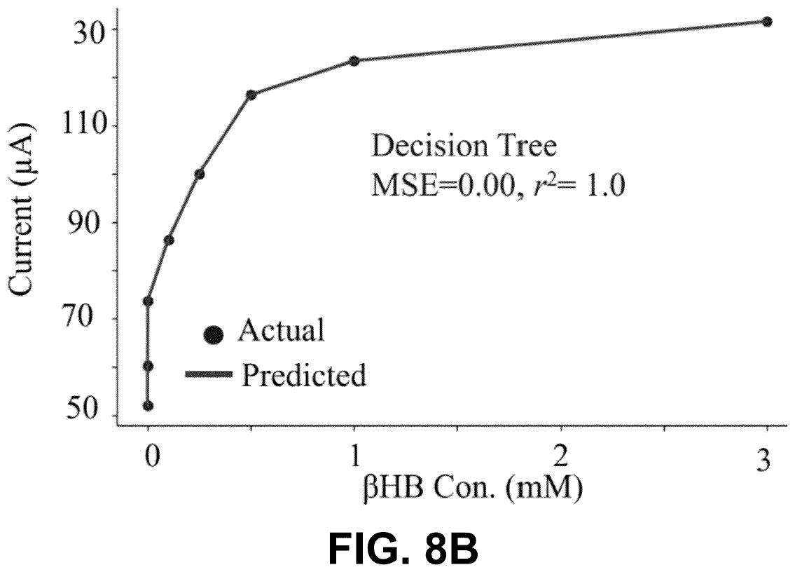

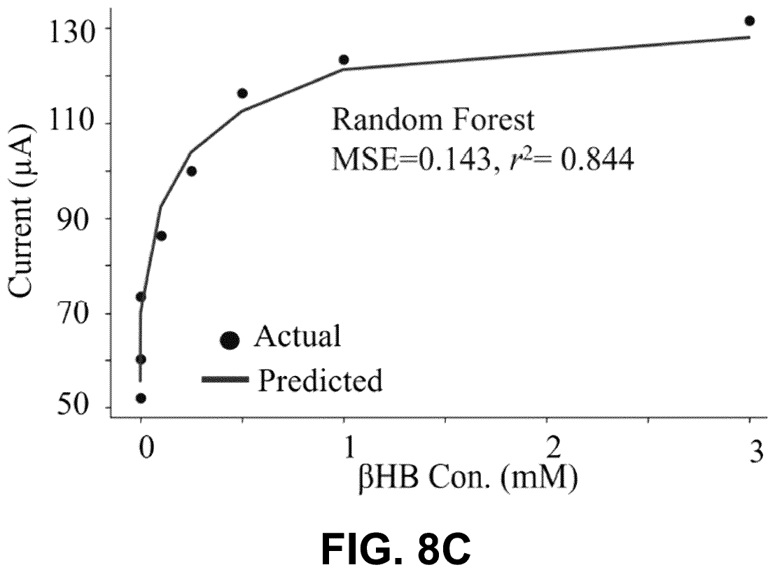

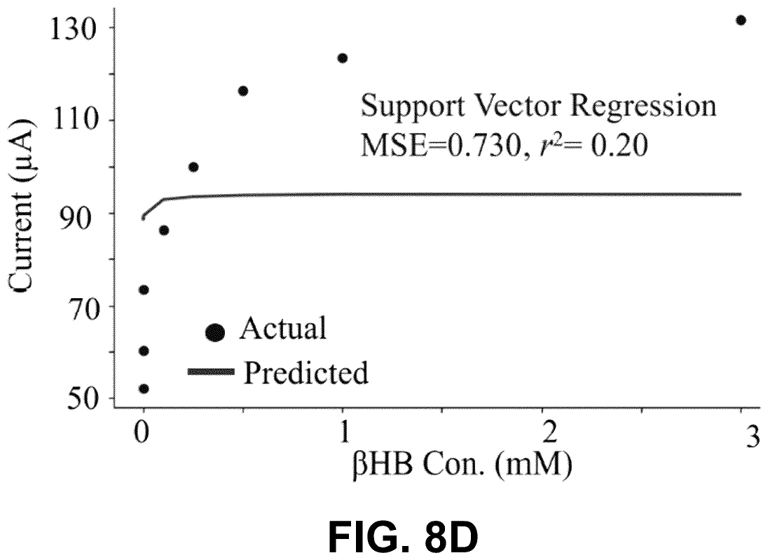

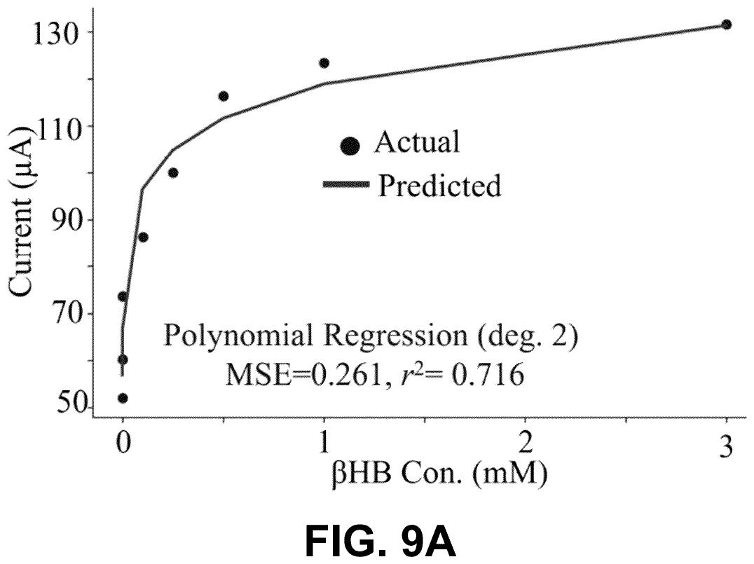

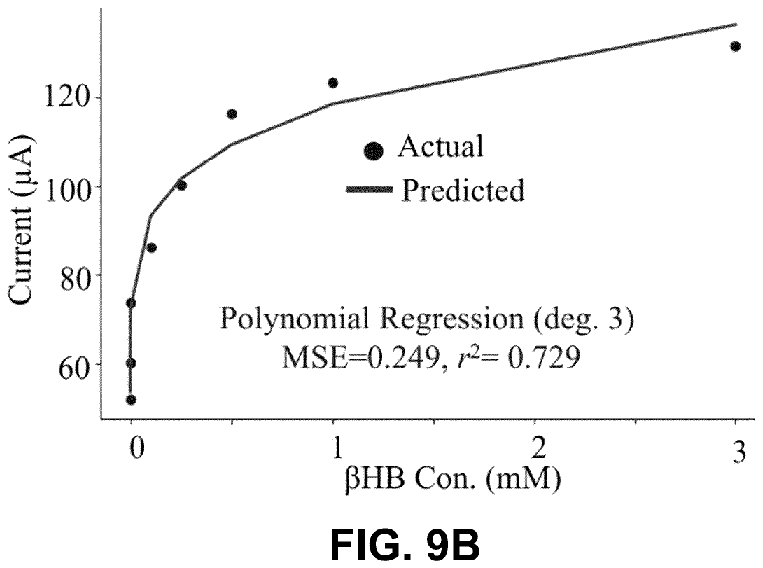

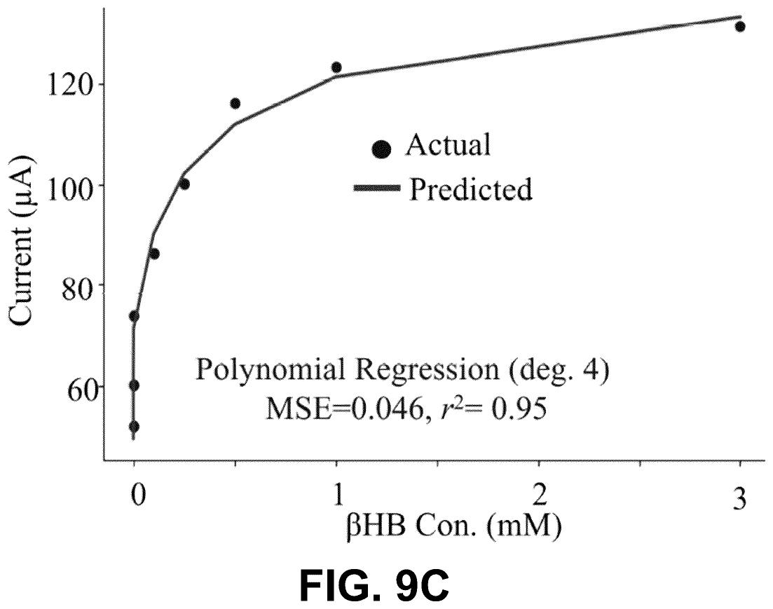

A comprehensive machine learning approach was employed to model the relationship between current measurements from a nanosensor and log-transformed βHB concentrations (obtained from FIG. 7). A number of regression models such as Linear Regression (LR) (FIG. 8A) for basic trend assessment, Non-linear Regression using Decision Trees (DT) (FIG. 8B), Random Forests (RF) (FIG. 8C), and Supporting Vector Regression (SVR) (FIG. 8D) to capture complex relationships, and Polynomial Regression (FIGS. 9A-9C) to address potential non-linearity. The performance of each model is evaluated through Mean Squared Error (MSE) and co-efficient of determination (r²) calculations, along with scatter plots for visual comparison. In the Linear Regression analysis (FIG. 8A), a poor fit was observed, particularly at higher concentrations of βHB, with noticeable discrepancies between the actual and predicted data. In contrast, among the non-linear regression models (FIGS. 8B-8D) such as RF, and SVR, the Decision Tree model demonstrated a perfect fit, evidenced by a Mean Squared Error (MSE) of 0.0 and an r² value of 1.0. With combining this machine learning approach, using this Decision Tree model is anticipated to enhance the accuracy of data prediction and the evaluation of unknown concentrations. The Decision Tree model's ability to capture complex patterns in the data is expected to yield more accurate predictions for unknown βHB concentrations.

In addition, a polynomial regression analysis was conducted to capture the curved relationships that arise when the relationship between variables is more complex than a straight line. Polynomial degrees of n (2, 3, and 4) were examined, which determine the shape of the curve (FIGS. 9A-9C). At a polynomial degree of 4, a nearly perfect fit was observed, as indicated by a low MSE of 0.046 and an r² value of 0.95, outperforming both degrees n=2 and n=3. This methodology allows for a robust comparison of linear and non-linear techniques, insights into scaling impacts, and a clearer understanding of the relationship dynamics in predicting ketone bodies or subclinical ketosis.

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Therefore, at least the following is claimed:

1. A system comprising:
a support;
a potentiostat;
a first electrode set into or layered on a surface of the support and in electrical communication with the potentiostat;
a second electrode set into or layered on a surface of the support and in electrical communication with the first electrode and the potentiostat; and
a third electrode set into or layered on a surface of the support and in electrical communication with the first electrode and the potentiostat;
wherein the first electrode comprises at least one layer of graphene oxide coated onto a surface of the electrode and an enzyme layer coated onto a surface of the graphene oxide layer, wherein the enzyme layer comprises β-hydroxybutyrate dehydrogenase (βHBD), β-nicotinamide adenine (NADH), and an enzyme-stabilizing agent.

2. The system of claim 1, wherein the enzyme layer is covalently bonded to the graphene oxide layer via an amide linkage between the graphene oxide layer and NADH.

3. The system of claim 1, wherein the enzyme-stabilizing agent is selected from glycerol, poly(GMA-ran-OEGMA), or a combination thereof.

4. The system of claim 1, wherein the system has a limit of detection of about 0.10 nM to about 0.30 nM for a target analyte.

5. The system of claim 4, wherein the target analyte is β-hydroxybutyrate.

6. An apparatus comprising:
a support;
a first electrode set into or layered on a surface of the support;
a second electrode set into or layered on a surface of the support; and
a third electrode set into or layered on a surface of the support;
wherein the first electrode comprises at least one layer of graphene oxide coated onto a surface of the electrode and an enzyme layer coated onto a surface of the graphene oxide layer, wherein the enzyme layer comprises β-hydroxybutyrate dehydrogenase (βHBD), β-nicotinamide adenine (NADH), and an enzyme-stabilizing agent.

7. The apparatus of claim 6, wherein the enzyme layer is covalently bonded to the graphene oxide layer via an amide linkage between the graphene oxide layer and NADH.

8. The apparatus of claim 6, wherein the enzyme-stabilizing agent is selected from glycerol, poly(GMA-ran-OEGMA), or a combination thereof.

9. A method for fabricating a sensor, comprising:
applying a first solution comprising graphene oxide nanosheets to an electrode;
drying the first solution and electrode, thereby forming a graphene oxide-coated electrode;
applying a second solution comprising N-Ethyl-N′-(3-dimethylaminopropyl) carbodiimide (EDC) and N-hydroxysuccinimide (NHS) to the graphene oxide-coated electrode, thereby forming a functionalized electrode; and
applying a third solution comprising an enzyme-stabilizing agent, β-hydroxybutyrate dehydrogenase (βHBD), and β-nicotinamide adenine (NADH) to the functionalized electrode.

10. The method of claim 9, wherein the EDC and NHS are present in the second solution in a weight ratio ranging from about 1:4 to about 4:1.

11. The method of claim 9, wherein the βHBD and NADH are present in the third solution in a weight ratio ranging from about 1:2 to about 2:1.

12. The method of claim 9, wherein the third solution comprises from about 5% to about 50% by volume of the enzyme-stabilizing agent.

13. The method of claim 9, wherein the first solution is applied to the graphene oxide-coated electrode at least one additional time prior to applying the second solution.

14. The method of claim 9, wherein the graphene oxide-coated electrode is washed prior to applying the third solution.

15. The method of claim 9, wherein the electrode is a carbon electrode.

16. The method of claim 9, wherein the enzyme-stabilizing agent is selected from glycerol, poly(GMA-ran-OEGMA), or a combination thereof.

17. A method, comprising:
obtaining a biological sample;
bringing the biological sample into contact with the first electrode, second electrode, and third electrode of the system of claim 1; and
measuring at least one of:
a) an open circuit potential between the first electrode and the third electrode; or
b) a current flow between the first electrode and the second electrode.

18. The method of claim 17, further comprising applying the measured current flow to a trained machine learning map, thereby generating a concentration of an analyte in the biological sample; wherein the machine learning map is generated by a machine learning model trained based on a known analyte concentration and a known current flow.

19. The method of claim 18, wherein the machine learning model is a machine learning regression model.

20. The method of claim 17, wherein the biological sample comprises a bovine biofluid.
